(19) United States
(12) Patent Application Publication
(10) Pub. No.: US 2024/0175056 A1
(43) Pub. Date: May 30, 2024

(54) PRIME EDITING-BASED SIMULTANEOUS GENOMIC DELETION AND INSERTION

(71) Applicant: UNIVERSITY OF MASSACHUSETTS, Boston, MA (US)

(72) Inventors: Tingting Jiang, Worcester, MA (US); Xiao-Ou Zhang, Shanghai (CN); Zhiping Weng, Wellesley Hills, MA (US); Wen Xue, Natick, MA (US)

(21) Appl. No.: 18/283,201

(22) PCT Filed: Mar. 15, 2022
(86) PCT No.: PCT/US22/20392

§ 371 Date: Sep. 20, 2023

Related U.S. Application Data
(60) Provisional application No. 63/165,487, filed on Mar. 24, 2021.

(51) Int. Cl. C12N 15/90; C12N 9/12; C12N 9/22; C12N 15/11

(52) U.S. Cl. CPC C12N 15/907; C12N 9/1276; C12N 9/22; C12N 15/11; C12Y 207/07049; C12N 2310/20; C12N 2310/3519; C12N 2310/531; C12N 2800/80

(57) ABSTRACT

Genomic insertions, duplications, and insertion/deletion (indels) account for ˜14% of human pathogenic mutations. Current gene editing methods cannot accurately or efficiently correct these abnormal genomic rearrangements, especially larger alterations (>100 bp). The presently disclosed compositions and methods accurately delete insertions/duplications and repair the deletion junction to improve the scope of gene therapies. For example, a Cas9 prime editor (PECas9) is combined with two prime editing guide RNAs (pegRNAs) targeting complementary DNA strands. PECas9 can replace an ˜1-kb genomic fragment with a desired sequence at the target site without requiring an exogenous DNA template.

### STATEMENT OF GOVERNMENT INTEREST

This invention was made with government support HL137167, HL131471 and HL147367, HL 153940 awarded by The National Institutes of Health. The government has certain rights in the invention.

### FIELD OF THE INVENTION

The present invention is related to the field of genetic engineering. In particular, in regards to methods and compositions to correct genomic mutations that are associated with diseases or other medical disorders. For example, a modified prime editor is used to delete and insert large polynucleotide sequences that is beyond the capability of conventional prime editors. The presently disclosed Cas9 prime editor is catalytically active, whereas conventional prime editors utilize a Cas9 nickase. The improved prime editor permits a therapeutic deletion/insertion event to treat diseases and medical disorders that are beyond the capability of conventional prime editors.

### BACKGROUND

Correction of genetic mutations in vivo is believed to have broad potential therapeutic application for a range of human genetic diseases. Prime editors (PE) composed of a Cas9 nickase and an engineered reverse transcriptase have been reported to result in nucleotide changes, sequence insertions and deletions. Anzalone et al., “Search-and-replace genome editing without double-strand breaks or donor DNA” Nature 576:149-157 (2019). PE does not induce double-stranded DNA breaks and does not require a donor DNA template in conjunction with homology directed repair.

Genomic insertions, duplications, and insertion/deletions (indels) may account for ˜14% of human pathogenic mutations. Current gene editing methods cannot accurately or efficiently correct these abnormal genomic rearrangements, especially larger alterations (e.g., >100 bp). Thus, what is needed in the art are compositions and methods to accurately delete large insertions/duplications and repair a deletion junction which improve the scope of gene therapies.

### SUMMARY

The present invention is related to the field of genetic engineering. In particular, in regards to methods and compositions to correct genomic mutations that are associated with diseases or other medical disorders. For example, a modified prime editor is used to delete and insert large polynucleotide sequences that is beyond the capability of conventional prime editors. The presently disclosed Cas9 prime editor is catalytically active, whereas conventional prime editors utilize a Cas9 nickase. The improved prime editor permits a therapeutic deletion/insertion event to treat diseases and medical disorders that are beyond the capability of conventional prime editors.

In one embodiment, the present invention contemplates a method, comprising: a) providing; i) a genomic DNA locus comprising a target nucleotide sequence; and ii) a composition comprising a catalytically active Cas9 protein fused to a reverse transcriptase, a first prime editor guide RNA (pegRNA) molecule conjugated to a first reverse transcriptase DNA insertion template and a second prime editor guide RNA molecule conjugated to a second reverse transcriptase DNA insertion template, wherein said first and second reverse transcriptase DNA templates are complementary; b) contacting said catalytically active Cas9 protein with said target nucleotide sequence, wherein said first pegRNA molecule binds to a sense strand of said target nucleotide sequence and said second pegRNA molecule binds to an antisense strand of said target nucleotide sequence; c) creating two double strand breaks in said target nucleotide sequence with said catalytically active Cas9 protein such that said target nucleotide sequence is deleted; and d) incorporating a double stranded insertion nucleotide sequence encoded by said first and second reverse transcriptase insertion templates into said genomic DNA sequence. In one embodiment, the target nucleotide sequence ranges between 1 kb to 10 kb. In one embodiment, the insertion nucleotide sequence has a length of up to 60 bp. In one embodiment, the target nucleotide sequence is linked to a genetic disease. In one embodiment, the genetic disease is tyrosinemia I. In one embodiment, the target nucleotide sequence comprises a Fah^{ΔExon 5} mutation.

In one embodiment, the present invention contemplates a method, comprising: a) providing; i) a patient exhibiting at least one symptom of a genetic disease; and ii) a composition comprising a catalytically active Cas9 protein fused to a reverse transcriptase, a first prime editor guide RNA (pegRNA) molecule conjugated to a first reverse transcriptase DNA insertion template and a second prime editor guide RNA molecule conjugated to a second reverse transcriptase DNA insertion template, wherein said first and second reverse transcriptase DNA templates are complementary; b) administering said composition to said patient such that said at least one symptom of said genetic disease is reduced. In one embodiment, the genetic disease is tyrosinemia. In one embodiment, the genetic disease is Huntington disease. In one embodiment, the patient further comprises a gene mutation insertion between 1 kb-10 kb. In one embodiment, the administering replaces said gene mutation insertion with an insertion nucleotide sequence that has a length of up to 60 bp.

In one embodiment, the present invention contemplates a composition comprising a catalytically active Cas9 protein fused to a reverse transcriptase, a first prime editor guide RNA (pegRNA) molecule conjugated to a first reverse transcriptase DNA template and a second prime editor guide RNA molecule conjugated to a second reverse transcriptase DNA template, wherein said first and second reverse transcriptase DNA templates are complementary. In one embodiment, the first reverse transcriptase DNA template is conjugated as a 3′ extension to the first pegRNA molecule. In one embodiment, the second reverse transcriptase DNA template is conjugated as a 3′ extension to the second pegRNA molecule. In one embodiment, the first and second reverse transcriptase DNA templates have a length of up to 60 bp.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” or “approximately” as used herein, in the context of any of any assay measurements refers to +/−5% of a given measurement.

As used herein, the term “CRISPRs” or “Clustered Regularly Interspaced Short Palindromic Repeats” refers to an acronym for DNA loci that contain multiple, short, direct repetitions of base sequences. Each repetition contains a series of bases followed by the same series in reverse and then by 30 or so base pairs known as “spacer DNA”. The spacers are short segments of DNA from a virus and may serve as a ‘memory’ of past exposures to facilitate an adaptive defense against future invasions (PMID 25430774).

As used herein, the term “Cas” or “CRISPR-associated (cas)” refers to genes often associated with CRISPR repeat-spacer arrays (PMID 25430774).

As used herein, the term “Cas9” refers to a nuclease from Type II CRISPR systems, an enzyme specialized for generating double-strand breaks in DNA, with two active cutting sites (the HNH and RuvC domains), one for each strand of the double helix. Jinek combined tracrRNA and crRNA (spacer RNA) into a “single-guide RNA” (sgRNA) molecule that, mixed with Cas9, could find and cleave DNA targets through Watson-Crick pairing between the guide sequence within the sgRNA and the target DNA sequence (PMID 22745249). There have been substantial efforts to broaden the targeting specificity of SpyCas9 through mutations that increase the number of PAMs that can be recognized. Two of the most prominent modified versions of Cas9 are xCas9 (Hu et al. 2018 (PMID 29512652)) and Cas9-NG (Nishimasu et al. 2018 (PMID 30166441)), both of which permit targeting some additional PAM elements.

As used herein, the term “guide RNA” refers to an RNA that programs a CRISPR-Cas protein to recognize a target site in the genome. This could be a crRNA, crRNA/tracrRNA, sgRNA or a pegRNA depending on the type of Cas9 protein and the modifications that have been made to the protein to incorporate extra functionality.

As used herein, the term “catalytically active Cas9” refers to an unmodified Cas9 nuclease comprising full nuclease activity.

The term “nickase” as used herein, refers to a nuclease that cleaves only a single DNA strand, either due to its natural function or because it has been engineered to cleave only a single DNA strand. Cas9 nickase variants (e.g. nSpCas9, nCas9) that have either the RuvC or the HNH domain mutated provide control over which DNA strand is cleaved and which remains intact (Jinek, et al. 2012 (PMID 22745249) and Cong, et al. 2013 (PMID 23287718)).

The term “protospacer adjacent motif” (or PAM) as used herein, refers to a DNA sequence that may be required for a Cas9/sgRNA to form an R-loop to interrogate a specific DNA sequence through Watson-Crick pairing of its guide RNA with the genome. The PAM may comprise a trinucleotide sequence having a single G residue (e.g., a single G PAM), or a trinucleotide sequence having two consecutive G residues (e.g., a dual G PAM). The PAM specificity may be a function of the DNA-binding specificity of the Cas9 protein (e.g., a “protospacer adjacent motif recognition domain” at the C-terminus of Cas9).

As used herein, the term “sgRNA” refers to single guide RNA used in conjunction with CRISPR associated systems (Cas). sgRNAs are a fusion of crRNA and tracrRNA and contain nucleotides of sequence complementary to the desired target site (Jinek, et al. 2012 (PMID 22745249)). Watson-Crick pairing of the sgRNA with the target site permits R-loop formation, which in conjunction with a functional PAM permits DNA cleavage or in the case of nuclease-deficient Cas9 allows binds to the DNA at that locus.

The term “primer binding site” as used herein, refers to a specific nucleic acid sequence within the pegRNA that is complementary to the 3′ or 5′ end of a cleaved target nucleotide sequence. This allows annealing of the free 3′ end or free 5′ end of the genomic DNA for extension by the reverse transcriptase based on the reverse transcriptase template sequence encoded in the pegRNA.

The term, “prime editing guide RNA molecule” or “pegRNA molecule” as used herein, refers to a Cas9 guide RNA molecule that encodes the crRNA-tracrRNA fused to a primer binding site (PBS) and a reverse transcriptase template (RTT). The primer binding site hybridizes to a desired genomic sequence released by the binding and cleavage of the Cas9 nickase. The 3′ end and/or 5′ end of a genomic sequence is extended by the reverse transcriptase based on the reverse transcriptase template sequence.

The term “prime editing” as used herein, is a genome editing technology by which the genome of living organisms may be modified. Prime editing manipulates the genetic information of a targeted DNA site to essentially “rewrite” the coded sequences.

The term “prime editor” or “PE” as used herein, is a fusion protein comprising a catalytically impaired Cas9 endonuclease (nickase; nCas9) that can nick DNA fused to an engineered reverse transcriptase enzyme, and a prime editing guide RNA (pegRNA). The pegRNA is capable of programming the nCas9 to recognize a target site with the encoded crRNA-tracrRNA. The resulting nicked genomic DNA can be extended by the reverse transcriptase based on the pegRNA template sequence to integrate a new sequence. Once one strand is recoded, cellular DNA repair pathways fill in the other strand to create the new sequence. Such manipulation includes, but is not limited to, insertions, deletions, and base-to-base conversions without the need for double strand breaks (DSBs) or donor DNA templates. For example, such prime editing may be performed by a Cas9 CRISPR platform programmed with a pegRNA, such as a catalytically impaired Cas9 nickase platform with an appropriate reverse transcriptase.

The term “base pairs” as used herein, refer to specific nucleobases (also termed nitrogenous bases), that are the building blocks of nucleotide sequences that form a primary structure of both DNA and RNA. Double stranded DNA may be characterized by specific hydrogen bonding patterns, base pairs may include, but are not limited to, guanine-cytosine and adenine-thymine) base pairs.

As used herein, the term “edit” “editing” or “edited” refers to a method of altering a nucleic acid sequence of a polynucleotide (e.g., for example, a wild type naturally occurring nucleic acid sequence or a mutated naturally occurring sequence) by selective deletion of a specific genomic target, the specific inclusion of new sequence through the use of an exogenously supplied DNA template, or the conversion of one DNA base to another DNA base.

Such a specific genomic target includes, but may be not limited to, a chromosomal region, mitochondrial DNA, a gene, a promoter, an open reading frame or any nucleic acid sequence.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “associated with” as used herein, refers to an art-accepted causal relationship between a genetic mutation and a medical condition or disease. For example, it is art-accepted that a patient having an HTT gene comprising a tandem CAG repeat expansion mutation has, or is a risk for, Huntington's disease.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “administered” or “administering”, as used herein, refers to any method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository etc.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “protein” as used herein, refers to any of numerous naturally occurring extremely complex substances (as an enzyme or antibody) that consist of amino acid residues joined by peptide bonds, contain the elements carbon, hydrogen, nitrogen, oxygen, usually sulfur. In general, a protein comprises amino acids having an order of magnitude within the hundreds.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The terms “Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand.

The term “antisense strand” as used herein, refers to a non-coding DNA strand of a gene. A cell uses antisense DNA strand as a template for producing messenger RNA (mRNA) that directs the synthesis of a protein.

The term “sense strand” as used herein, refers to a coding DNA strand of a gene. A cell uses sense DNA strand to encode the associated amino acid sequence of a protein.

The term “an isolated nucleic acid”, as used herein, refers to any nucleic acid molecule that has been removed from its natural state (e.g., removed from a cell and is, in a preferred embodiment, free of other genomic nucleic acid).

The terms “amino acid sequence” and “polypeptide sequence” as used herein, are interchangeable and to refer to a sequence of amino acids.

The term “portion” when used in reference to a nucleotide sequence refers to fragments of that nucleotide sequence. The fragments may range in size from 5 nucleotide residues to the entire nucleotide sequence minus one nucleic acid residue.

A “deletion” is defined as a change in either nucleotide or amino acid sequence in which one or more nucleotides or amino acid residues, respectively, are absent.

An “insertion” or “addition” is that change in a nucleotide or amino acid sequence which has resulted in the addition of one or more nucleotides or amino acid residues.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “C-A-G-T,” is complementary to the sequence “G-T-C-A.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

The terms “homology” and “homologous” as used herein in reference to nucleotide sequences refer to a degree of complementarity with other nucleotide sequences. There may be partial homology or complete homology (i.e., identity). A nucleotide sequence which is partially complementary, i.e., “substantially homologous,” to a nucleic acid sequence is one that at least partially inhibits a completely complementary sequence from hybridizing to a target nucleic acid sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding the probe will not hybridize to the second non-complementary target.

The terms “homology” and “homologous” as used herein in reference to amino acid sequences refer to the degree of identity of the primary structure between two amino acid sequences. Such a degree of identity may be directed a portion of each amino acid sequence, or to the entire length of the amino acid sequence. Two or more amino acid sequences that are “substantially homologous” may have at least 50% identity, preferably at least 75% identity, more preferably at least 85% identity, most preferably at least 95%, or 100% identity.

An oligonucleotide sequence which is a “homolog” is defined herein as an oligonucleotide sequence which exhibits greater than or equal to 50% identity to a sequence, when sequences having a length of 100 bp or larger are compared.

As used herein, the term “hybridization” is used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the Tₘ of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C₀ t or R₀ t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the term “an oligonucleotide having a nucleotide sequence encoding a gene” means a nucleic acid sequence comprising the coding region of a gene, i.e. the nucleic acid sequence which encodes a gene product. The coding region may be present in a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the terms “nucleic acid molecule encoding”, “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the term “gene” means the deoxyribonucleotide sequences comprising the coding region of a structural gene and including sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogeneous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation

### BRIEF DESCRIPTION OF THE FIGURES

FIG. 1 presents a PEDAR system that mediates a large sequence deletion and a simultaneous sequence insertion at an endogenous genomic locus.

FIG. 1A: Classification of the 60,008 known human pathogenic genetic variants reported in the ClinVar database¹.

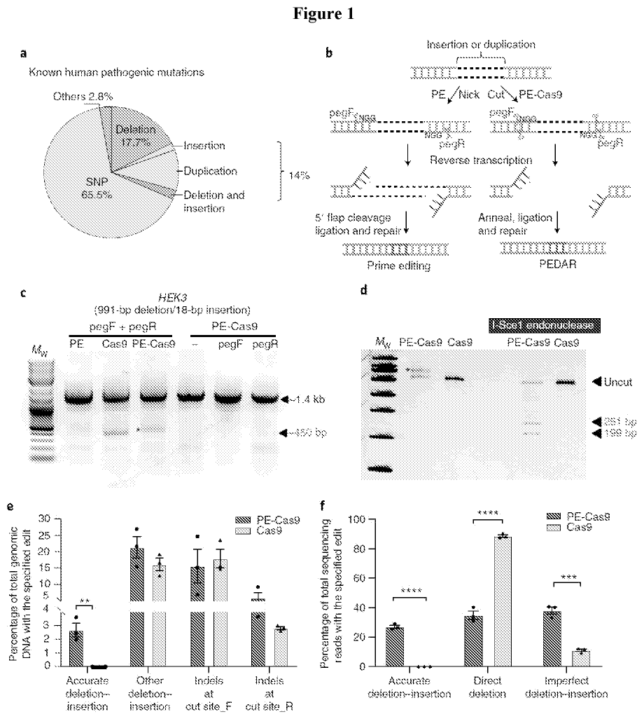

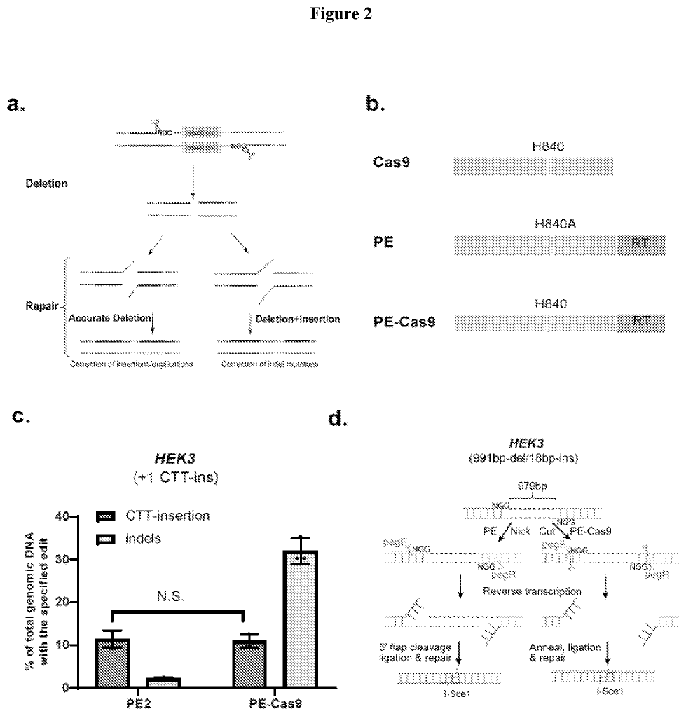

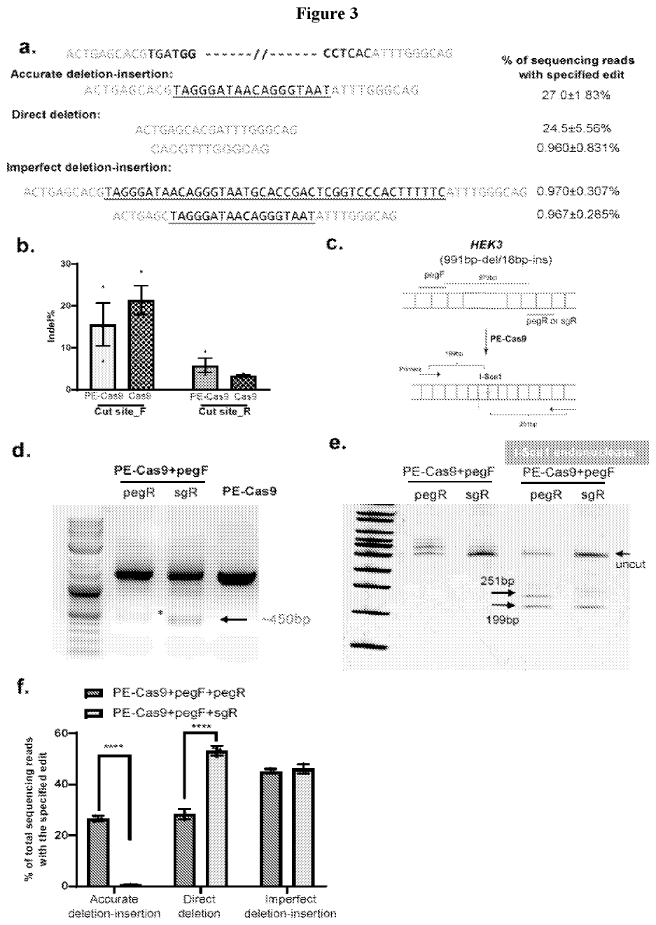

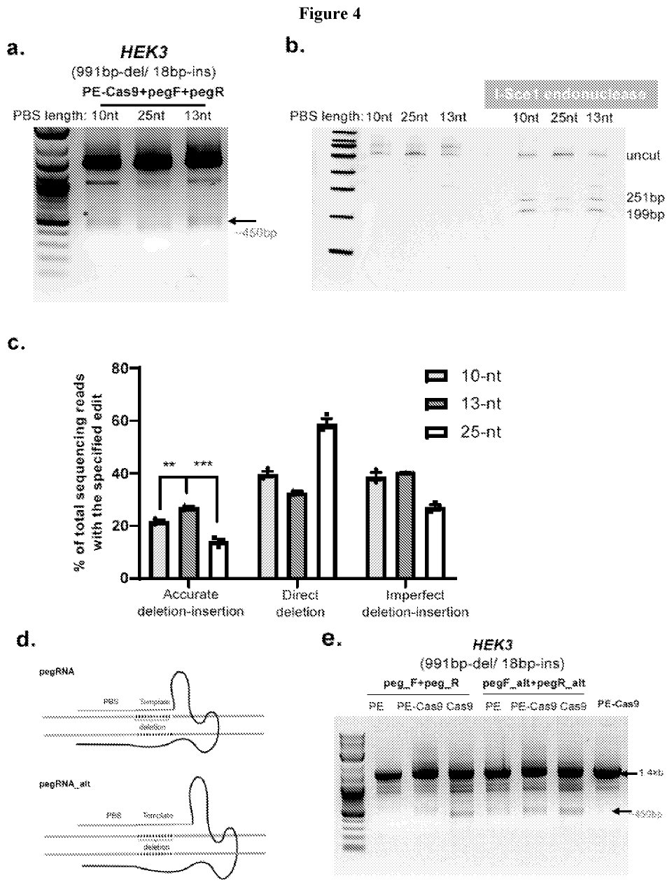

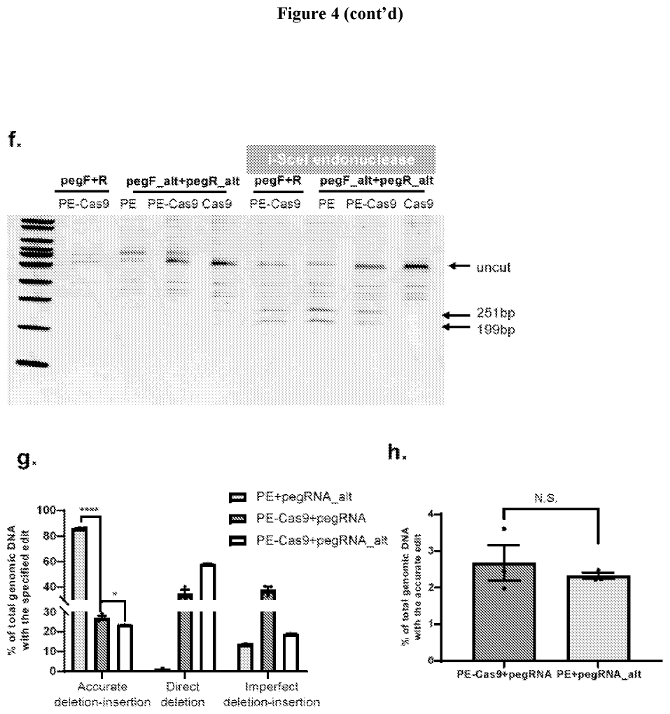

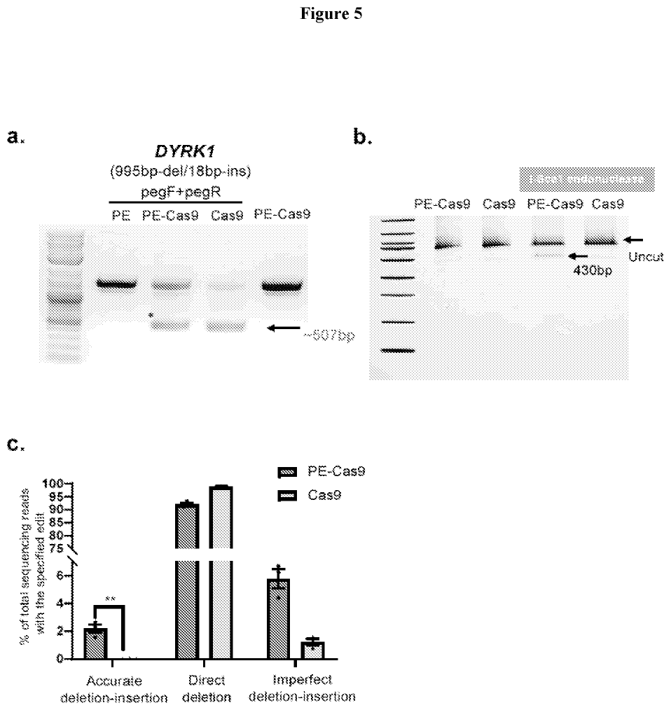

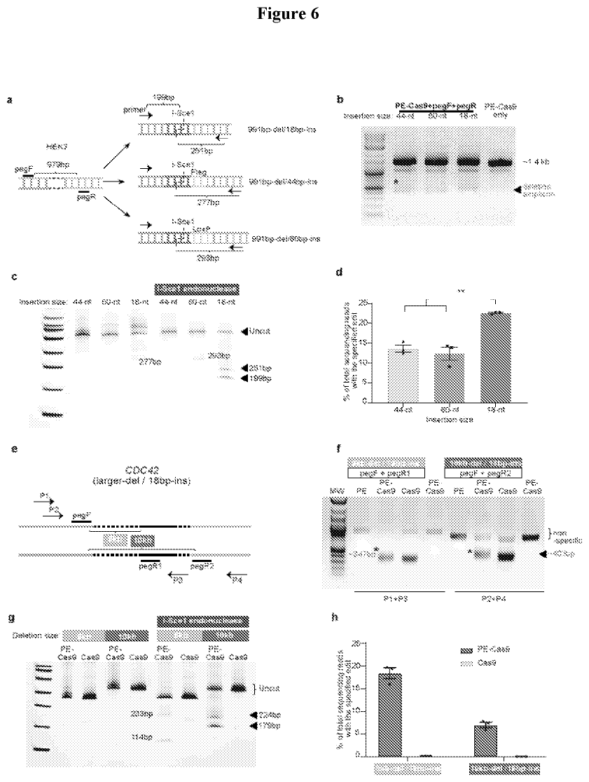

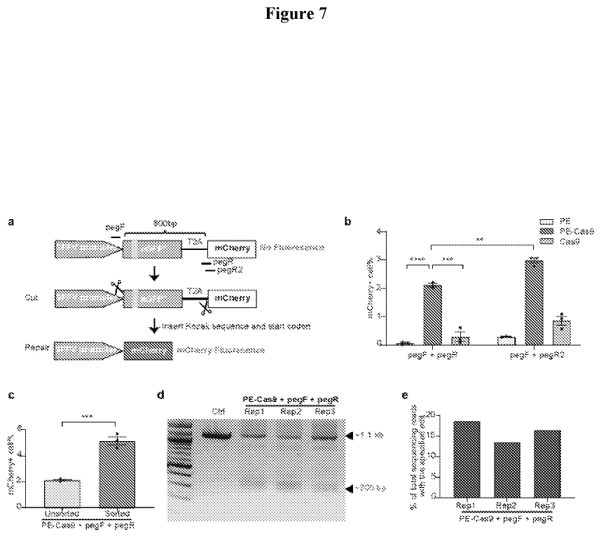

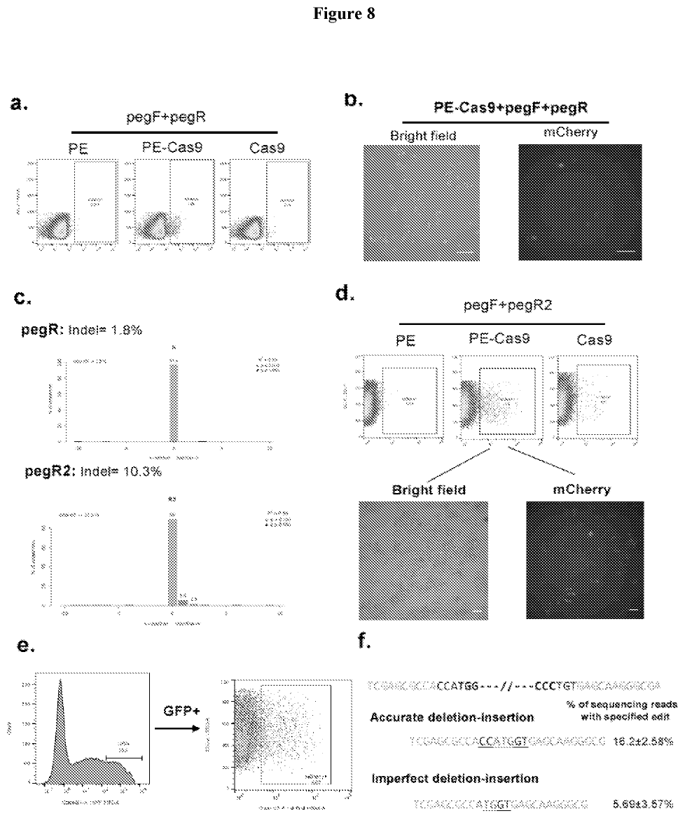

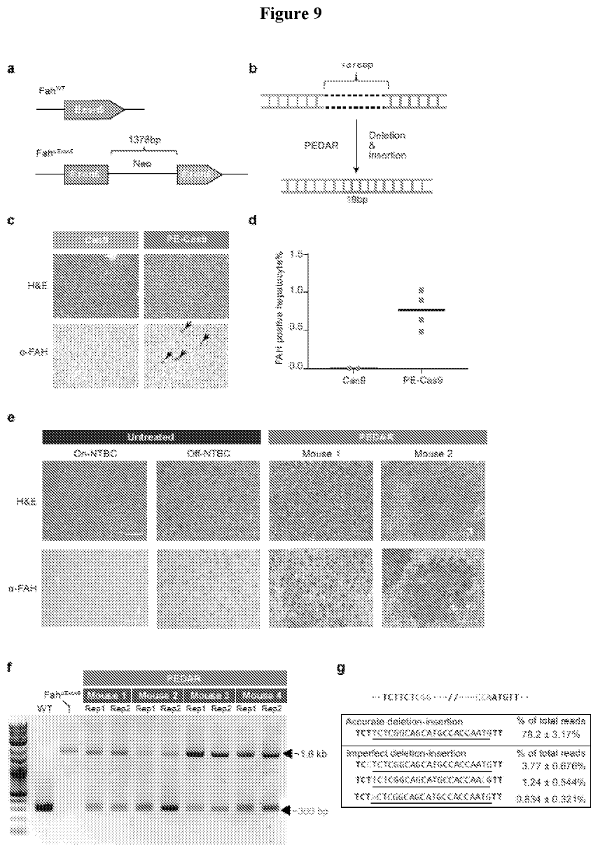

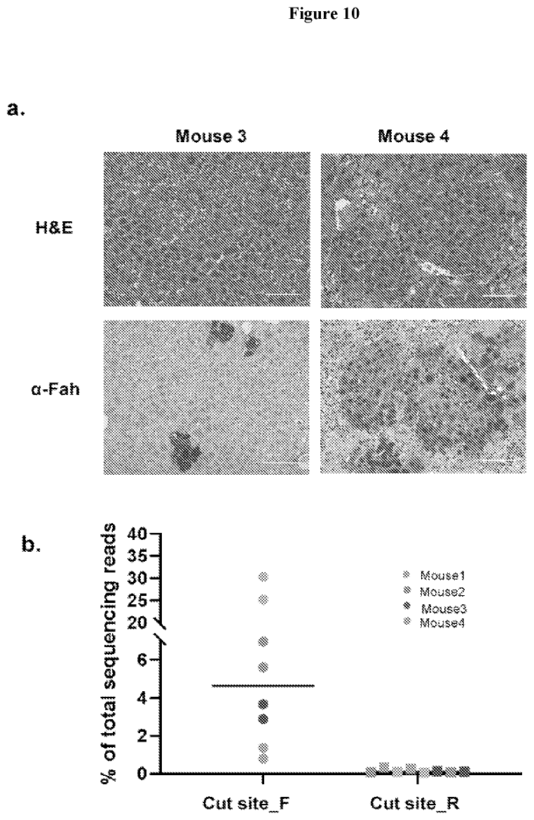

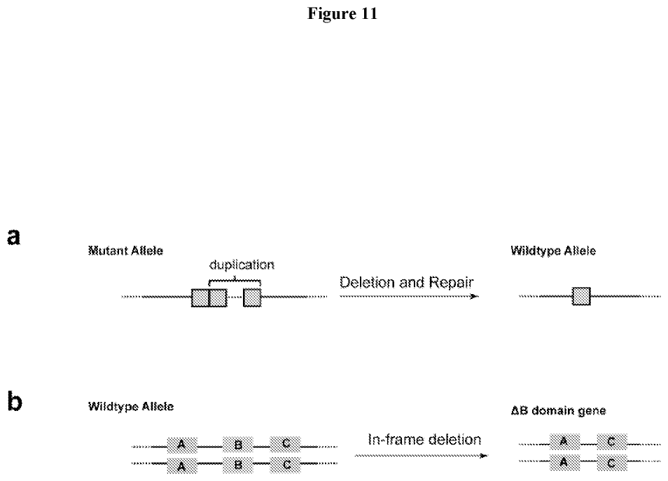

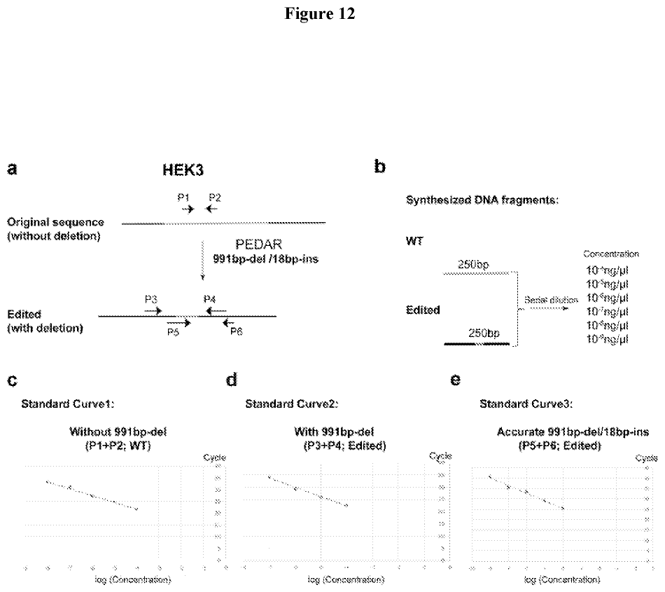

FIG. 1B: Overview of using prime editing (left) and PEDAR (right) to generate accurate deletion-insertion.
Conventional Prime Editing: Two PE complexes—consisting of pegRNA (pegF or pegR) and a Cas9 nickase (Cas9 H840A) conjugated to an engineered reverse transcriptase (RT)—recognize ‘NGG’ PAM sequences, bind, and nick the target DNA strands. After hybridization of the nicked DNA strand to the primer binding sequence of pegRNA, the desired edit is reverse transcribed into the target site using the RT template at the 3′ extension of pegRNA. The desired inserted edits (red) at the two nicking sites are complementary. After equilibration between the edited 3′ flap and the unedited 5′ flap, the 5′ flap is cleaved, and DNA repair results in coupled deletion and insertion of target sequence.
PEDAR: Dual PECas9: pegRNA (pegF or pegR) complexes recognize ‘NGG’ PAM sequences, bind, and cut the target DNA. The two complementary desired edits (red) are reverse transcribed into the target sites using the RT template at the 3′ extension of pegRNAs. The inserted sequences are annealed, and the double stranded DNA break is repaired

FIG. 1C: Deleting a 991-bp DNA fragment and simultaneous insertion of I-Sce1 recognition sequence (18 bp) at the HEK3 locus (Chr9:107422166-107423588). Target genomic region was amplified using primers that span the cut sites. The paired pegRNAs targeting complementary DNA strand are denoted as pegF and pegR. HEK293T cells were transfected with PE, Cas9, or PE-Cas9 with or without single or paired pegRNAs. The ˜450-bp band is the expected deletion amplicon (denoted with *), and the ˜1.4-kb band is the amplicon without deletion.

FIG. 1D: Deletion amplicons from Cas9- or PE-Cas9-treated groups shown in FIG. 1C were incubated with or without I-SceI endonuclease and analyzed in 4-20% TBE gel. Digested products are marked by arrows with expected sizes. Original amplicon is marked as “uncut”. The band with insertion of i-Sce1 recognition sequence is denoted with *.

FIG. 1E: PE-Cas9 or Cas9-mediated absolute rates of all the editing events in total genomic DNA at HEK3 site. Data represent mean±SEM (n=3 biologically independent samples). P=0.0053 (**).

FIG. 1F: Deep Sequencing of Deletion Amplicons Shown in FIG. 1C. Bar Chart shows distribution of all deletion events, including accurate deletion/insertion, direct deletion (deletion without any insertions), or imperfect deletion-insertion. Editing rate=the reads with indicated editing/total deletion events. Data represent mean±SEM (n=3 biologically independent samples). P=<0.0001 (****), 0.002 (***), two-tailed t-test.

FIG. 2 presents exemplary data showing correction of pathogenic mutations with a PEDAR system.

FIG. 2A: Proposed model of CRISPR-associated gene correction of pathogenic mutations caused by insertions/duplications or indels. The pathogenic insertion is removed by CRISPR under the guidance of dual sgRNAs targeting two complementary strands of DNA, while the repair or insertion is concurrently performed at the cut site.

FIG. 2B: PECas9 is engineered by replacing the Cas9^{H840A} nickase (nCas9) in a conventional PE platform with a catalytically active Cas9 nuclease.

FIG. 2C: Comparison of PE2(nCas9)- and PECas9-mediated insertion of a 3-bp nucleotide sequence (“CTT”) at the nicking or cut site of the HEK3 locus. HEK293T cells were transfected with a pegRNA and PECas9 or conventional PE. The rate of accurate insertion and indels was assessed by deep sequencing. P=0.7929 (Not significant, N.S.), two-tailed t-test.

FIG. 2D: Diagram of concurrent deletion of a 991-bp DNA fragment and insertion of 18-bp I-SceI recognition sequence (red) by conventional PE or PECas9 with paired pegRNAs. Two pegRNAs having an offset of 979 bp (distance between the two ‘NGG’ PAM sequences) were designed and transfected with either conventional PE or PECas9 into cells.

FIG. 3 presents exemplary data showing deep sequencing of insertion sequences by a PEDAR system.

FIG. 3A: PECas9-mediated editing events with highest reads across three replicates by deep sequencing. The two PAM sequences are in bold, and the original sequences before or after the two cut sites are highlighted in blue and green. The inserted sequence is underlined. Data represent mean±SD (n=3 biologically independent samples).

FIG. 3B: The indel rates generated by individual pegRNA at the two cut sites of HEK3 site, hereafter referred to as cut site_F and cut site_R, assessed by Sanger sequencing. Data represent mean±SEM (n=3 biologically independent samples).

FIG. 3C: Diagram showing that treatment of the edited PCR product with I-Sce1 endonuclease would lead to two DNA fragments of 199-bp and 251-bp at length.

FIG. 3D: Amplification of target genomic region using primers that span the cut sites at HEK3 locus. HEK293T cells were transfected with PE-Cas9, pegF, and pegR or sgR. The ˜450-bp band is the deletion amplicon. Cells transfected with PE-Cas9 alone serve as negative control.

FIG. 3E: Deletion amplicons from pegR or sgR-treated groups shown in FIG. S2D were incubated with or without I-SceI endonuclease and analyzed in 4-20% TBE gel. The digested products are marked by arrows with expected sizes. The original amplicon is marked as “uncut”.

FIG. 3F: Deep sequencing of deletion amplicons shown in FIG. S2D. Bar chart shows distribution of all the deletion events, including accurate deletion-insertion, direct deletion (deletion without any insertions), or imperfect deletion-insertion. Data represent mean±SEM (n=3 biologically independent samples). P=<0.0001 (****), two-tailed t-test.

FIG. 4 presents exemplary data showing PEDAR activity using various lengths of primer binding site sequences and reverse transcriptase template sequences in a pegRNA.

FIG. 4A: Amplification of a target genomic region using primers that span the cut sites at HEK3 site. Paired pegRNAs with indicated lengths of primer binding site sequence were designed and transfected with PECas9 into HEK293T cells. The ˜450-bp band (denoted with *) is the expected deletion amplicon.

FIG. 4B: Deletion amplicons from groups shown in FIG. S3A were incubated with or without I-SceI 19 endonuclease and analyzed in 4-20% TBE gel. The digested products are marked with expected sizes. The original amplicon is marked as “uncut”.

FIG. 4C: Deep sequencing of deletion amplicons shown in FIG. S3A. Bar chart shows distribution of all the deletion events, including accurate deletion-insertion, direct deletion (deletion without any insertions), or imperfect deletion-insertion. Data represent mean±SEM (n=3 biologically independent samples). P=0.0046 (**), =0.0004 (***), two-tailed t-test.

FIG. 4D: Design alternative pegRNA (pegRNA_alt) by extending an RT template (RTT) with a 14-nt sequence homologous to the region after the other cut site.

FIG. 4E: Amplification of a target genomic region using primers that span the cut sites at the HEK3 locus. HEK293T cells were transfected with Cas9, PECas9 or conventional PE along with paired pegRNAs as indicated. The ˜450-bp band is the expected deletion amplicon. Cells transfected with PECas9 alone serve as negative control.

FIG. 4F: Deletion amplicons from groups shown in FIG. S3E were incubated with or without I-SceI endonuclease and analyzed in 4-20% TBE gel. The digested products are marked by arrows with expected sizes. The original amplicon is marked as “uncut”.

FIG. 4G: Deep sequencing of deletion amplicons shown in FIG. S3E. Bar chart shows distribution of all deletion events, including accurate deletion-insertion, direct deletion (deletion without any insertions), or imperfect deletion-insertion. Data represent mean±SEM (n=3 biologically independent samples). P=<0.0001 (****), =0.0269 (*), two-tailed t-test.

FIG. 4H: Absolute accurate editing rates of PEDAR (PECas9+pegRNA) and PRIME-Del (conventional PE+pegRNA_alt) at the HEK3 locus calculated by quantitative PCR in total genomic DNA. P=0.5156 (N.S., not significant).

FIG. 5 presents exemplary data showing PEDAR activity at a DYRK1 locus.

FIG. 5A: Amplification of a target genomic region using primers that span the cut sites at a DYRK1 locus. The paired pegRNAs targeting complementary DNA strand are denoted as pegF and pegR. HEK293T cells were transfected with conventional PE, conventional Cas9, or PECas9 with or without paired pegRNAs. The size of deletion amplicon (denoted with *) is indicated.

FIG. 5B: Deletion amplicons from groups shown in FIG. S4A were incubated with or without I-SceI endonuclease and analyzed in 4-20% TBE gel. The digested products are marked by arrows with expected sizes. The original amplicon is marked as “uncut”.

FIG. 5C: Deep sequencing of deletion amplicons shown in FIG. S4A. Bar chart shows distribution of all the deletion events, including accurate deletion-insertion, direct deletion (deletion without any insertions), or imperfect deletion-insertion. Data represent mean±SEM (n=3 biologically independent samples). P=0.0036 (**), two-tailed t-test.

FIG. 6 presents exemplary data showing the flexibility of PEDAR systems in programming larger sequence deletions and sequence insertions as compared to conventional PE and conventional Cas9 platforms.

FIG. 6A: Insert DNA sequences of variable lengths (18-bp, 44-bp, and 60-bp) to a target site of the HEK3 locus. pegRNAs and primers for amplifying the target site are as shown. The expected sizes of digestion products after ISce1 treatment are shown.

FIG. 6B: Amplification of a target genomic region using primers spanning the cut sites at HEK3 locus. HEK293T cells were transfected with PE-Cas9 and paired pegRNAs. The deletion amplicons are denoted with *. Cells transfected with PE-Cas9 alone serves as negative control.

FIG. 6C: Deletion amplicons from groups shown in FIG. 2B were incubated with or without I-SceI endonuclease and analyzed in 4-20% TBE gel. Digested products are marked by arrows with expected sizes. The original amplicon is marked as “uncut”.

FIG. 6D: Deep sequencing of deletion amplicons shown in FIG. 2B. Bar chart shows accurate deletion-insertion rate. Data represent mean±SEM (n=3 biologically independent samples). P=0.0024 (**), two-tailed t-test.

FIG. 6E: Test of the efficiency of PEDAR in mediating larger deletions. Paired pegRNAs spaced ˜8-kb (pegF+pegRl) or 10-kb (pegF+pegR2) apart were designed as indicated to target the CDC42 locus. Primers used to amplify the target genomic regions are as marked (P1+P3 and P2+P4).

FIG. 6F: Target genomic region was amplified using the primers indicated in FIG. 2E. Dual pegRNAs were transfected into HEK293T cells with PE, Cas9, or PE-Cas9. Cells transfected with PE-Cas9 alone serve as negative control. The deletion amplicons are marked with expected sizes (denoted with *).

FIG. 6G: Deletion amplicons from Cas9- or PE-Cas9-treated groups shown in FIG. 2F were incubated with or without I-SceI endonuclease and analyzed in 4-20% TBE gel. Digested products are marked with expected sizes. The original amplicon is marked as “uncut”.

FIG. 6H: Deep sequencing of deletion amplicons shown in FIG. 2F. Bar chart shows rate of accurate deletion-insertion events. Data represent mean±SEM (n=3 biologically independent samples).

FIG. 7 presents exemplary data showing that a PEDAR system generates in-frame deletions to restore mCherry expression in TLR reporter cells.

FIG. 7A: Diagram of a TLR reporter system. GFP sequence is disrupted by an insertion (grey). Deleting the disrupted GFP sequence and inserting Kozak sequence and start codon will restore mCherry protein expression.

FIG. 7B: TLR reporter cells transfected with indicated paired pegRNAs along with PE, PE-Cas9, or Cas9 were analyzed by flow cytometry, and the percentage of mCherry positive cells are shown among different groups. Data represent mean±SEM (n=3 biologically independent samples). P<0.0001 (****), =0.0004 (***), =0.0015 (**), two-tailed t-test.

FIG. 7C: mCherry positive cell rate before and after sorting of cells with high transfection level. A plasmid expressing GFP was co-transfected with paired pegRNAs and PECas9 into TLR cells. Three days later, cells with high GFP expression were selected for analyzing mCherry signal by flow cytometry. Data represent mean±SEM (n=3 biologically independent samples). P=0.0007 (***), two-tailed t-test.

FIG. 7D: TLR reporter cells edited by PEDAR were selected by flow cytometry (for mCherry signal) and subjected to PCR amplification using primers spanning the two cut sites. The amplicon with the desired deletion is ˜300 bp compared to a ˜1.1-kb PCR products in control group. Rep: replicate; Ctrl: untreated TLR reporter cells.

FIG. 7E: Efficiency of accurate deletion-insertion in three PEDAR-edited replicates (Rep 1-3) measured by deep sequencing of the deletion amplicons shown in FIG. 3D.

FIG. 8 presents exemplary data of a PEDAR system using a traffic light reporter (TLR) model.

FIG. 8A: A representative flow cytometry plot shows the gating of mCherry positive cells in conventional PE-, PECas9-, or conventional Cas9-treated groups.

FIG. 8B: Image of mCherry positive cells treated by a PEDAR system. Scale bar=100 μm.

FIG. 8C: TIDE results showing the indels introduced by two distinct pegRNAs (pegR and pegR2) at a TLR locus. Cas9 was transfected together with pegR or pegR2 in HEK293T cells. Indel rates were analyzed by Tide software (tide.nki.nl).

FIG. 8D: Upper panel: representative flow cytometry plot shows the gating of mCherry positive cells in conventional PE, PECas9, or conventional Cas9-treated groups. Lower panel: image of mCherry positive cells treated by PEDAR. Scale bar=100 m.

FIG. 8E: Flow cytometry plots show the gating of TLR cells with high GFP expression (left panel; ˜20% of total population) and the gating of mCherry positive cell after sorting out the GFP positive cells (right panel). GFP expression serves as an indicator of transfection rate.

FIG. 8F: The rate of accurate editing and the most common imperfect deletion-insertion editing events identified across three replicates. The two PAM sequences are in bold, and the original sequences before or after the two cut sites are highlighted in blue and green. The inserted sequence is underlined. Start codon is highlighted in red. Data represent mean±SD (n=3 biologically independent samples).

FIG. 9 presents exemplary data showing that a PEDAR system corrects a pathogenic mutation insertion in a Tyrosinemia I Fah^{ΔExon5} mouse model.

FIG. 9A: The Tyrosinemia I Fah^{ΔExon5} mouse model was derived by integrating a ˜1.38-kb neo expression cassette at exon 5 of the Fah gene.

FIG. 9B: Diagram showing the application of PEDAR to delete the ˜1.38-kb insertion and concurrently repair the target region by inserting a 19-bp DNA fragment (marked in red).

FIG. 9C: Immunohistochemistry staining and Hematoxylin and Eosin staining (H&E) of mouse liver sections seven days after injection of dual pegRNAs with Cas9 or PE-Cas9. FAH protein positive hepatocytes are pointed by arrows. Mice were kept on NTBC until being euthanized. Scale bar=100 m.

FIG. 9D: Quantification of FAH protein expressing hepatocytes shown in FIG. 9C. n=2 (Cas9-treated group), n=4 (PECas9-treated group).

FIG. 9E: Immunohistochemistry and H&E staining of mouse liver sections 40 days after injection of PE-Cas9 with dual pegRNAs. Mice (n=4) were kept off NTBC. Mouse 1 and 2 denote two representative mice from the treatment group.

The liver sections from untreated Fah^{ΔExon5} mice kept on or off NTBC serve as negative controls. Scale bar=100 m.

FIG. 9F: Amplification of exon 5 of Fah gene from mouse livers 40 days post injection of PECas9 and paired pegRNAs. The corrected amplicon size is around ˜300 bp, compared to a ˜1.6-kb amplicon without deletion. Four mice in treated group and two liver lobes (denoted as Rep 1 and 2) per mouse were analyzed. WT: wild type C57BL/6J mouse. Fah^{ΔExon5}: untreated Fah^{ΔExon5} mouse.

FIG. 9G: Accurate correction rate and the top-three imperfect editing events identified by deep sequencing. Two PAM sequences are in blue and green. The 22-bp intended insertion (19-bp deletion fragment plus a 3-bp unintentionally deleted sequence) is underlined. Mutated nucleotides in imperfect editing sequences are highlighted in yellow. Data represent mean±SD (n=8, two liver lobes/mouse; four mice in total).

FIG. 10 presents exemplary data showing PEDAR activity in a Tyrosinemia I mouse model.

FIG. 10A: Immunohistochemistry staining and Hematoxylin and Eosin staining (H&E) of mouse liver sections 40 days after injection of PECas9 with dual pegRNAs. Mice were kept off NTBC after treatment until euthanizing. Mouse 3 and 4 denote two distinct mice from the treatment group. Scale bar=100 m.

FIG. 10B: Indel rates generated by individual pegRNA at the two cut sites at the Fah locus. Four mice in treated group and two liver lobes per mouse were analyzed. The dots with the same color indicate samples from two liver lobes of the same mouse.

FIG. 11 illustrates alternative uses for a PEDAR system.

FIG. 11A: Correction of large pathogenic mutations and/or chromosomal aberrations such as duplicated sequences.

FIG. 11B: In-frame deletions to study the functional domain of a protein.

FIG. 12 presents exemplary amplification of the edited target site.

FIG. 12A: Design of three pairs of qPCR primers to amplify the target site at the HEK3 locus.

FIG. 12B: Design of two 250-bp DNA fragments (denoted as “WT” and “Edited”) of the same sequence with unedited or accurately edited target site.

FIG. 12C: A standard curve reflecting the correlation between qPCR cycle number and the concentration of DNA without the 991-bp deletion.

FIG. 12D: A standard curve reflecting the correlation between qPCR cycle number and the concentration of DNA with the 991-bp deletion.

FIG. 12E: A standard curve reflecting the correlation between qPCR cycle number and the concentration of DNA with the accurate 991-bp deletion/18-bp insertion.

### DETAILED DESCRIPTION OF THE INVENTION

The present invention is related to the field of genetic engineering. In particular, in regards to methods and compositions to correct genomic mutations that are associated with diseases or other medical disorders. For example, a modified prime editor is used to delete and insert large polynucleotide sequences that is beyond the capability of conventional prime editors. The presently disclosed Cas9 prime editor is catalytically active, whereas conventional prime editors utilize a Cas9 nickase. The improved prime editor permits a therapeutic deletion/insertion event to treat diseases and medical disorders that are beyond the capability of conventional prime editors.

In one embodiment, the present invention contemplates a Cas9 prime editor (PECas9) comprising a catalytically active Cas9 nuclease conjugated to a reverse transcriptase and combined with two prime editing guide RNAs (pegRNAs) having complementary reverse transcriptase template nucleotide strands. Although it is not necessary to understand the mechanism of an invention, it is believed that PECas9 can replace a genomic fragment, ranging from to ˜1 Kb to >10 Kb, with any desired sequence without requiring an exogenous DNA template.

This system, designated herein as a “PECas9-Based Deletion And Repair” (PEDAR) system has been shown herein to restore mCherry expression through an in-frame deletion of a disrupted green fluorescent protein (GFP) DNA sequence. Further shown is that PEDAR efficiency is enhanced by using pegRNAs with high cleavage activity or increasing transfection efficiency. In tyrosinemia mice, a PEDAR system removed a 1.38-kb pathogenic insertion within the Fah gene and precisely repaired the deletion junction to restore FAH protein expression in liver. These data demonstrate that PECas9 compositions and PEDAR methods can be an efficacious clinical therapy for correcting pathogenic mutations by replacing large nucleotide sequences and/or chromosomal aberrations.

In one embodiment, the present invention contemplates compositions and methods to perform precise genome editing that accurately deletes insertion/duplication mutations of DNA sequences and repairs the disrupted genomic site to treat a wide range of diseases.

### I. Conventional Prime Editor Complexes

Genetic insertions, duplications, and indels (insertion/deletion) account for ˜14% of 60,008 known human pathogenic variants. See, FIG. 1A. Many of these abnormal insertions and duplications involve large DNA fragments (e.g., >100 bp). Indeed, retrotransposon element insertions range from 163 to 6000 bp. Cordaux et al., “The impact of retrotransposons on human genome evolution” Nat Rev Genet 10:691-703 (2009); and Chen et al., “A systematic analysis of LINE-1 endonuclease dependent retrotranspositional events causing human genetic disease” Hum Genet 117:411-427 (2005). Such large genetic aberrations disrupt the normal expression and function of genes thereby causing genetic diseases like cystic fibrosis, hemophilia A, X-linked dystonia-parkinsonism, and inherited cancers. Hancks et al., “Roles for retrotransposon insertions in human disease” Mobile DNA 7:9 (2016); Wang et al., “Human Retrotransposon Insertion Polymorphisms Are Associated with Health and Disease via Gene Regulatory Phenotypes” Front Microbiol 8:1418 (2017); Hancks et al., “Active human retrotransposons: variation and disease” Curr Opin Genet Dev 22:191-203 (2012); and Qian et al., “Identification of pathogenic retrotransposon insertions in cancer predisposition genes” Cancer Genet 216-217:159-169 (2017).

The CRISPR/Cas9 system is a proposed gene editing tool for correcting pervasive pathogenic gene mutations. When using dual single guide RNAs (sgRNA), Cas9 is believed to induce two double-strand breaks (DSBs). The two cut ends can then be ligated through the non-homologous end joining (NHEJ) repair pathway, leading to <5-Mb target fragment deletion in vitro and in vivo. Ran et al., “Double nicking by RNA-guided CRISPR Cas9 for enhanced genome editing specificity” Cell 154:1380-1389 (2013); Cong et al., “Multiplex genome engineering using CRISPR/Cas systems” Science 339:819-823 (2013); Kato et al., “Creation of mutant mice with megabase-sized deletions containing custom-designed breakpoints by means of the CRISPR/Cas9 system” Sci Rep 7:59 (2017); Hara et al., “Microinjection-based generation of mutant mice with a double mutation and a 0.5 Mb deletion in their genome by the CRISPR/Cas9 system” J Reprod Dev 62:531-536 (2016); and Wang et al., “Large genomic fragment deletion and functional gene cassette knock-in via Cas9 protein mediated genome editing in one-cell rodent embryos” Sci Rep 5:17517 (2015).

However, the random indels generated by NHEJ lower the editing accuracy of this method. When a donor DNA template is present, CRISPR/Cas9 can insert a desired sequence at the cut site to repair the deletion junction through homology directed repair (HDR). Yeh et al., “Advances in genome editing through control of DNA repair pathways” Nat Cell Biol 21:1468-1478 (2019). This method has been used successfully in precise gene deletion and replacement application. Zheng et al., “Precise gene deletion and replacement using the CRISPR/Cas9 system in human cells” Biotechniques 57:115-124 (2014). Nevertheless, the repair efficiency of CRISPR-mediated HDR is hindered by the exogenous DNA donor and is limited in post-mitotic cells. Cox et al., “Therapeutic genome editing: prospects and challenges” Nature Medicine 21:121-131 (2015); and Liu et al., “Methodologies for Improving HDR Efficiency” Front Genet 9:691 (2018).

To further expand the gene editing toolbox, a CRISPR-associated gene editor—called prime editing (PE)—was developed by conjugating an engineered reverse transcriptase (RT) to a catalytically-impaired Cas9 ‘nickase’ (Cas9^{H840A}) that cleaves only one DNA strand. An extension at the 3′ end of the prime editing guide RNA (pegRNA) encodes an RT template, allowing the nicked site to be precisely repaired. Anzalone et al., “Search-and-replace genome editing without double-strand breaks or donor DNA” Nature 576:149-157 (2019); and Matsoukas, I. G., “Prime Editing: Genome Editing for Rare Genetic Diseases Without Double-Strand Breaks or Donor DNA” Front Genet 11:528 (2020).

Thus, conventional PE complexes can mediate small deletions, small insertions, and limited base editing without creating double stranded DNA breaks or requiring donor DNA. Schene et al., “Prime editing for functional repair in patient-derived disease models” Nat Commun 11:5352 (2020); Jiang et al., “Prime editing efficiently generates W542L and S621I double mutations in two ALS genes in maize” Genome Biology 21:257 (2020); Liu et al., “Improved prime editors enable pathogenic allele correction and cancer modelling in adult mice” bioRxiv, 2020.2012.2015.422970 (2020); Jang, H. et al., “Prime editing enables precise genome editing in mouse liver and retina” bioRxiv 2021.2001.2008.425835 (2021). Yet, conventional PE has been unsuccessfully applied to delete large DNA sequences.

Conventional PE complexes are constructed with a nicking Cas9, one pegRNA and one nicking gRNA. If one of skill would consider using a conventional prime editor complex with two prime editing guide RNAs (pegRNAs), an attempt to replace large genomic DNA sequences might be outlined as follows (see, FIG. 1C):
(i) recognizing and hybridizing a dual pegRNA nCas9 system to a DNA target site with flanking ‘NGG’ PAM sequences;
(ii) nicking each complementary strand of DNA on either side of the large fragment with the nCas9 (Ran et al., “Double nicking by RNA-guided CRISPR Cas9 for enhanced genome editing specificity” Cell 154; 1380-1389 (2013);
(ii) reverse transcribing two insertion sequences linked to each of the pegRNAs into the nicked target site;
(iii) annealing the complementary DNA strands containing the insertion sequence;
(iv) excising the original (replaced) DNA sequences (i.e., 5′ flaps); and
(v) repairing the DNA target site.

However, this theoretical modification of a conventional prime editor complex is considered in the art not to have a reasonable expectation of success because it has been reported that a prime editor Cas9 nickase complex is not effective in mediating larger target deletions with paired guide RNAs. Song et al., “CRISPR-Cas9(D10A) Nickase-Assisted Genome Editing in Lactobacillus casei” Appl Environ Microbiol 83 (2017); and Cho et al., “Analysis of off-target effects of CRISPR/Cas-derived RNA-guided endonucleases and nickases” Genome Res 24; 132-141 (2014). Indeed, PE applications reported in the literature are limited to programing deletions of less than 100 bp, raising the concern that PE cannot generate long genomic deletions. Matsoukas I. G., “Prime Editing: Genome Editing for Rare Genetic Diseases Without Double-Strand Breaks or Donor DNA” Front Genet 11:528 (2020).

### II. PECas9-Based Deletion/Insertion (PEDAR) Complexes

To achieve accurate and efficient large nucleotide sequence deletion and simultaneous nucleotide sequence insertion, without requiring a DNA template, a conventional prime editing system was improved by using a catalytically active Cas9 nuclease with a pair of pegRNAs (hereafter referred to as pegF and pegR) rather than a nickase Cas9 with one pegRNA and one nicking guide RNA. See, FIG. 2A. Using two pegRNAs enables concurrent targeting of both DNA strands. As a 3′ extension of each pegRNA is conjugated a reverse-complementary RT template, which encode the double stranded insertion nucleotide sequence.

This newly-engineered system can mediate an accurate deletion/insertion repair through the following exemplary steps: (i) prime editor recognizes the ‘NGG’ PAM sequence, binds, and cleaves both complementary strands of DNA on either side of the large sequence⁸; (ii) the encoded insertion sequences are then reverse transcribed between the cleavage sites of the complementary strands using the RT template linked to the pegRNAs; (iii) the complementary DNA strands containing the insertion sequence are annealed; (iv) the original DNA strands (i.e., 5′ flaps) are excised; and (v) the DNA is repaired by endogenous DNA repair pathways. See, FIG. 1B, left side. However, a conventional Cas9 nickase cannot effectively mediate large target deletions (e.g., >500 bp) even with paired guide RNAs^{23, 24}. Indeed, conventional PE applications are generally reported in the literature as limited to programing deletions of less than 100 bp, raising the concern that a conventional PE platform cannot generate long genomic deletions¹⁷.

### A. PECas9 Construction And Validation

Catalytically active Cas9 nuclease has been used to program larger deletions with dual conventional sgRNAs¹⁴. In one embodiment, the present invention contemplates a primer editor composition comprising a catalytically active Cas9 nuclease (instead of a conventional PE Cas9 nickase) that is conjugated to a reverse transcriptase (RT) to create “PECas9”. See, FIG. 2B. When using a single pegRNA both PECas9 and conventional PE generated similar rates of a 3-bp CTT deletion/insertion at an endogenous locus, indicating that catalytically active Cas9 nuclease activity does not affect prime editing efficiency¹⁷. See, FIG. 2C.

Although it is not necessary to understand the mechanism of an invention, it is believed that when two pegRNAs target both complementary strands of DNA, PECas9 introduces two DSBs and deletes an intervening DNA fragment between the two DSBs. Concurrently, an insertion nucleotide sequence is incorporated at the deletion site using the respective RT templates conjugated as a 3′ extension on each of the two pegRNAs. The two complementary insertion sequences then function as a homologous sequence to induce an endogenous ligation and repair of the deletion junction. See FIG. 1B, right side.

The efficiencies of PEDAR systems, conventional PE systems, and conventional Cas9 systems were compared for large deletion sequences coupled together with an accurate large insertion sequence at an endogenous HEK3 genomic locus in HEK293T cells. For this comparison, two pegRNAs were designed with an offset of 979 bp (e.g., the distance between the two ‘NGG’ PAM sequences) to program a 991 bp deletion sequence with an 18 bp insertion sequence at the HEK3 site. The 3′ extension RT template of the pegRNAs encoded an I-SceI recognition sequence (18-bp), which was reversed transcribed and integrated into the deletion site. See, FIG. 2D.

The two pegRNAs were transfected into cells along with a conventional PE, a PECas9, or a conventional Cas9. Delivery of PECas9 with or without a single pegRNA was used as a negative control and the target site was amplified three days post-transfection. The data showed that either PECas9 or a conventional Cas9, but not a conventional PE, led to a ˜450-bp deletion amplicon. The conventional PE amplicon was ˜1-kb shorter than the amplicon without a deletion. See, FIG. 1C.

Deletion amplicons from each group were digested with I-SceI endonuclease, and it was observed that only PECas9 showed cut bands of expected size (˜251 bp and ˜199 bp), indicating insertion of the I-SceI recognition sequence. See, FIG. 1D. Using real-time quantitative PCR, it was found that PECas9 generates an accurate deletion/insertion frequency of 2.67±0.839% in total genomic DNA, whereas a conventional Cas9 seldom generated an accurate deletion/insertion, as demonstrated by a frequency of 0.0112±0.00717%. See, FIG. 1E. To further verify gene editing accuracy, deletion amplicons were digested and deep sequencing analysis was performed. The data shows that PECas9 mediates 27.0±1.83% accurate editing of total deletion events. See, FIG. 1F. Taken together, these findings suggest that, PEDAR outperforms conventional prime editing and conventional Cas9 editing in achieving accurate large fragment sequence deletions with simultaneous replacement sequence insertions.

The PEDAR system also generated unintended edits, classified as: (i) other deletions/insertions, including a direct deletion without insertion and imperfect deletion/insertions, and (ii) small indels generated by individual pegRNA at the two cut sites, hereafter referred to as cut site_F and cut site_R. The incidence of these unintended events was measured in total genomic DNA by real-time quantitative PCR, and it was observed that PECas9 and conventional Cas9 generated comparable rates of unintended edits. See, FIG. 1E. A deep sequencing analysis of these events showed that PECas9 generated 38.0±4.15% imperfect deletion/insertions caused by imprecise DNA repair or improper pegRNA scaffold insertion. A significantly lower rate of PECas9 direct deletion without insertion was observed than that mediated by conventional Cas9 (35.0±4.80% and 88.8±1.58%, respectively). See, FIG. 1F.

PECas9-mediated unintended deletion edits with the highest sequencing reads were evaluated. See, FIG. 3A and Table 1.

TABLE 1
Representative Unintended Deletion Edits

| Aligned Sequence | Editing Type | % Total Reads |
|---|---|---|
| Replicate 1 | | |
| TTGGGGCCCAGACTGAGCACG--------- | Accurate deletion without | 23.70678 |
| ---------ATTTGGGCAGGTGATCAATGC | insertion | |
| TTGGGGCCCAGACTGAGCACGTAGGGATAAC | Inaccurate deletion-insertion | 1.190254 |
| AGGGTAATGCACCGACTCGGTCCCACTTTTTC | | |
| ATTTGGGCAGGTGATCAATGC | | |
| TTGGGGCCCAGACTGAGCACGTAGGGATAAC | Inaccurate deletion-insertion | 1.048521 |
| AGGGTAATGCACCGACTCGGTCCCACTTTTAT | | |
| TTGGGCAGGTGATCAATGC | | |
| TTGGGGCCCAGACTGAGCACGTAGGGATAAC | Inaccurate deletion-insertion | 0.990989 |
| AGGGTAATGCACCGACTCGGTCCCACTTTTTC | | |
| AATGC | | |
| TTGGGGCCCAGACTGAGCACGTAGGGATAAC | Inaccurate deletion-insertion | 0.971939 |
| AGGGTAATGCACCGACTCGGTCCCACTTTTTC | | |
| AAGTTATTTGGGCAGGTGATCAATGC | | |
| TTGGGGCCCAGACTGAGCACG--- | Inaccurate deletion-insertion | 0.879355 |
| GGATAACAGGGTAATATTTGGGCAGGTGATC | | |
| AATGC | | |
| TTGGGGCCCAGACTGAGCACGT---------- | Inaccurate direct deletion | 0.872878 |
| --------TTGGGCAGGTGATCAATGC | | |
| TTGGGGCCCAGACTGAGCACGCTAGGGATAA | Inaccurate deletion-insertion | 0.750957 |
| CAGGGTAATATTTGGGCAGGTGATCAATGC | | |
| TTGGGGCCCAGACTGAGC----- | Inaccurate deletion-insertion | 0.632846 |
| TAGGGATAACAGGGTAATATTTGGGCAGGTG | | |
| ATCAATGC | | |
| Replicate_2 | | |
| TTGGGGCCCAGACTGAGCACG--------- | Accurate deletion without | 30.04403 |
| ---------ATTTGGGCAGGTGATCAATGC | insertion | |
| TTGGGGCCCAGACTGAGCACGTAGGGATAAC | Accurate deletion-insertion | 26.54179 |
| AGGGTAATATTTGGGCAGGTGATCAATGC | | |
| TTGGGGCCCAGACTGAGCACGTAGGGATAAC | Inaccurate deletion-insertion | 1.955458 |
| AGGGTAATGCTAGGGATAACAGGGTAATATT | | |
| TGGGCAGGTGATCAATGC | | |
| TTGGGGCCCAGACTGAGCACGT--------- | Inaccurate direct deletion | 1.814373 |
| ---------TTGGGCAGGTGATCAATGC | | |
| TTGGGGCCCAGACTGAGC--- | Inaccurate deletion-insertion | 1.026402 |
| TAGGGATAACAGGGTAATATTTGGGCAGGTG | | |
| ATCAATGC | | |
| TTGGGGCCCAGACTGAGCACGTGCTAGGGAT | Inaccurate deletion-insertion | 0.732119 |
| AACAGGGTAATATTTGGGCAGGTGATCAATG | | |
| C | | |
| TTGGGGCCCAGACTGAGCACGTAGGGATAAC | Inaccurate direct deletion | 0.693988 |
| AGGGTAATGC-------------------- | | |
| TTGGGGCCCAGACTGAGCACGTAGGGATAAC | Inaccurate deletion-insertion | 0.634324 |
| AGGGTAATGCACCGACTCGGTCCCACTTTTT | | |
| CATTTGGGCAGGTGATCAATGC | | |
| TTGGGGCCCAGACTGAGCAC----------- | Inaccurate direct deletion | 0.602922 |
| --------ATTTGGGCAGGTGATCAATGC | | |
| Replicate_3 | | |
| TTGGGGCCCAGACTGAGCACGTAGGGATAAC | Accurate deletion-insertion | 29.0334 |
| AGGGTAATATTTGGGCAGGTGATCAATGC | | |
| TTGGGGCCCAGACTGAGCACG---------- | Accurate deletion without | 18.91839 |
| ---------ATTTGGGCAGGTGATCAATGC | insertion | |
| TTGGGGCCCAGACTGAGCACGTAGGGATAAC | Inaccurate deletion-insertion | 2.113317 |
| AGGGTAATGCTAGGGATAACAGGGTAATATT | | |
| TGGGCAGGTGATCAATGC | | |
| TTGGGGCCCAGACTGAGCACGGCTAGGGATA | Inaccurate deletion-insertion | 1.522825 |
| ACAGGGTAATATTTGGGCAGGTGATCAATGC | | |
| TTGGGGCCCAGACTGAGC--- | Inaccurate deletion-insertion | 1.217999 |
| TAGGGATAACAGGGTAATATTTGGGCAGGTG | | |
| ATCAATGC | | |
| TTGGGGCCCAGACTGAGCACGTAGGGATAAC | Inaccurate deletion-insertion | 1.100858 |
| AGGGTAATGCACCGACTCTAGGGATAACAGG | | |
| GTAATATTTGGGCAGGTGATCAATGC | | |
| TTGGGGCCCAGACTGAGCACGTAGGGATAAC | Inaccurate deletion-insertion | 1.039893 |
| AGGGTAATGCACCGACTCGGTCCCACTTTTTC | | |
| ATTTGGGCAGGTGATCAATGC | | |
| TTGGGGCCCAGACTGAGCACGTGCTAGGGAT | Inaccurate deletion-insertion | 1.020733 |
| AACAGGGTAATATTTGGGCAGGTGATCAATG | | |
| C | | |
| TTGGGGCCCAGACTGAGCACGTAGGGATAAC | Inaccurate deletion-insertion | 0.948881 |
| AGGGTAATGCACCGACTCGGTCCCACTTITTA | | |
| TTTGGGCAGGTGATCAATGC | | |
| TTGGGGCCCAGACTGAGCACGCTAGGGATAA | Inaccurate deletion-insertion | 0.90185 |
| CAGGGTAATATTTGGGCAGGTGATCAATGC | | |

PECas9 or conventional Cas9 also introduced indels at the two cut sites without generating the desired deletion. Sanger sequencing of these amplicons without a deletion reveals no significant difference in small indels caused by either PECas9 or conventional Cas9. See, FIG. 1C; ˜1.4-kb band; and FIG. 3B.

Potential repair mechanisms underlying PEDAR-mediated editing were evaluated by delivering PECas9 with one pegRNA and one sgRNA targeting the HEK3 locus and PECas9 with two pegRNAs. See, FIG. 3C. Although the pegRNA/sgRNA PECas9 system generated a ˜450-bp deletion amplicon, this amplicon failed to be digested into two distinct bands by I-Sce1 endonuclease. See, FIG. 3D and FIG. 3E, respectively. Deep sequencing revealed inaccurate deletion/insertion frequencies in the cells transfected with one pegRNA and one sgRNA (0.716±0.0868%), as compared to accurate deletion/insertion frequencies in the cells treated with two pegRNAs (26.5±1.12%). See, FIG. 3F. These data demonstrate that the introduction of two complementary and reverse sequences by two pegRNAs at a deletion site results in highly accurate repair, resembling an annealing and ligation process in endogenous MMEJ or SSA repair pathways^{25,26} Alternative designs of a primer binding site (PBS) and/or an RT template of PECas9 pegRNAs were evaluated for changes in PEDAR editing efficiency. pegRNAs were constructed with a 10-nt PBS, a 13-nt PBS or a 25-nt PBS targeting an HEK3 locus. Although all pegRNA lengths supported an ˜1-kb deletion and simultaneous insertion of the I-Sce1 recognition sequence, the 10-nt and 25-nt PBS lengths significantly impaired an accurate editing rate as identified by deep sequencing. See, FIGS. 4A-4C. To determine the effect of an RT template design on editing efficiency, an alternative pegRNA (pegRNA_alt) was constructed by extending the RT template with a 14-nt sequence homologous to a region after the cut site. See, FIG. 4D.

After transfecting a cell with a pegRNA_alt and either conventional PE or PECas9 a deletion amplicon of the expected size was identified and insertion of I-Sce1 recognition sequence was detected. See, FIGS. 4E and 4F. Deep sequencing revealed that pegRNA_alt significantly decreased PECas9-mediated accurate editing rates as compared to the original pegRNAs. See, FIG. 4G. Surprisingly, co-transfection of a conventional PE and pegRNA_alt greatly improved deletion product purity and editing accuracy (85.9±0.644%). See, FIG. 4G. However, an absolute accurate editing rate in total genomic DNA was comparable between conventional PE/pegRNA_alt and PECas9/original pegRNA groups, potentially due to the limited ability of Cas9 nickase to introduce larger deletion^{23, 24}. See, FIG. 4H. Based on these collective data, pegRNAs with a 13-nt PBS and an RT template without additional homologous sequences to a target site were deemed as optimal for PEDAR systems To assess the efficiency of PEDAR-mediated deletion/insertion at an endogenous locus other than a HEK3 site, a DYRK1 locus was targeted to delete a 995-bp DNA fragment and simultaneously insert an I-Sce1 recognition sequence. In HEK293T cells, a PEDAR system lead to a ˜507-bp deletion band and the amplified product was digested by I-Sce1 endonuclease. See, FIG. 5A and FIG. 5B, respectively. Deep sequencing of the deletion amplicon identified a 2.18±0.552% accurate editing efficiency. See, FIG. 5C. Although it is not necessary to understand the mechanism of an invention, it is believed that a low G/C content of pegRNA primer binding sequences targeting DYRK1 locus (23% of pegF and 31% of pegR) restricted the integration of the RTT sequence. These data are consistent with a report showing poor conventional PE efficiency when a PBS GC content is less than 30%.²⁷ The limits of PEDAR system deletion sequence and insertion sequence sizes were determined. An I-Sce1 recognition sequence was inserted into an HEK3 locus together with either a Flag epitope tag (44 bp total) or a Cre recombinase LoxP site (60 bp total) after deletion of a ˜1-kb DNA fragment. The pegRNAs were designed with either a nominal 18-nt RTT and compared to a 44-nt RTT or a 60-nt RTT. See, FIG. 6A. For all tested pegRNAs, the expected deletion sequence and the expected insertion sequence were observed at the target site in cells. See, FIGS. 6B and 6C, respectively. Deep sequencing revealed accurate deletion/insertion rates of 13.7±1.51% with a 44 bp-RTT and 12.4±2.88% with a 60 bp-RTT, which are significantly lower than a 22.6±0.267% accurate editing efficiency of the nominal 18 bp RTT. See, FIG. 6D.

To investigate a maximum deletion size for a PEDAR system, two sets of paired pegRNAs were designed with either an offset of ˜8 kb or ˜10 kb targeted at the CDC42 locus. See, FIG. 6E Amplification of the corresponding target site, the expected deletion amplicon was observed. See, FIG. 6F. After I-Sce1 endonuclease treatment, two digested bands were detected. See, FIG. 6B. Deep sequencing revealed accurate deletion/insertion rates of 18.4±2.07% for the 8 kb offset pegRNA and 6.97±1.00% for the 10 kb offset pegRNA. See, FIG. 6H. In all, these data demonstrate the robustness and flexibility of PECas9 in generating >10 kb deletion and up to a 60 bp insertion.

### B. PEDAR-Mediated Gene Function Restoration

A PEDAR system was validated to generate large in-frame deletions and accurately repair genomic coding regions to restore gene expression. A HEK293T traffic light reporter (TLR) cell line was used which contains a green fluorescent protein GFP sequence with an insertion and an mCherry sequence separated by a T2A (2A self-cleaving peptides) sequence^{28,29}. The TLR system generates a disrupted GFP sequence that causes a frameshift which prevents mCherry expression. See, FIG. 7A.

A PEDAR system was tested to restore an mCherry signal by accurately deleting a disrupted GFP and T2A sequence having ˜800 bp in length. Two pegRNAs were designed that targeted the GFP promoter region before the start codon and the site immediately after T2A, respectively. In this approach, part of the Kozak sequence and start codon were unintentionally deleted due to the restriction of the PAM sequence. However, the RT template at the 3′ end of pegRNAs was designed to encode missing the Kozak sequence and start codon to ensure their insertion into the target site by reverse transcription. See, FIG. 7A.

TLR reporter cells were treated with dual pegRNAs (e.g., pegF+pegR) and either PECas9, conventional PE, or conventional Cas9, and the mCherry signal were assessed by flow cytometry. The frequency of mCherry positive cells was significantly higher in the PECas9-treated group (2.12±0.105%) as compared to either the conventional PE or conventional Cas9 groups. See, FIG. 7B and FIGS. 8A, 8B. The mCherry positive cell rate was limited in all three replicates, likely because the cleavage efficiency of pegRNA at cut site_R (pegR) are very low (˜1.8%). See, FIG. 8C. Thus, another pegRNA (pegR2) was designed with a ˜10.3% cleavage rate. See, FIG. 7A and FIG. 8C. pegR2 significantly improved the mCherry positive cell rate (2.99±0.166%). See, FIG. 7B and FIG. 8D.

Alternatively, to enhance the editing rate, improving the expression level of gene editing agents in cells was evaluated.³⁰ Co-transfection of cells with a fluorescent protein-expressing plasmid, followed by FACS sorting, has been reported to enrich for cells with high levels of transgene expression^{31, 32}. Thus, a GFP-expressing plasmid was co-transfected with PECas9 and paired pegRNAs into TLR cells as an indicator of transfection efficiency. A ˜1.42-fold increase in mCherry positive cell rate was observed after selection of cells with high GFP expression. See, FIG. 7C and FIG. 8E. These results indicate that the editing efficiency of PEDAR systems may largely rely on the efficiency of pegRNA and the expression level of gene editing components.

To verify that PEDAR systems restore mCherry expression via accurate deletion-insertion, mCherry positive cells were sorted in PECas9-treated groups and the insertion sequences were amplified. The data shows a deletion amplicon that is ˜800-bp shorter than an amplicon in untreated control cells. See, FIG. 7D. Further, deep sequencing analysis of the ˜300-bp deletion amplicon revealed a 16.2±2.58% accurate deletion/insertion rate. See, FIG. 7E. The most common imperfect editing event across the three replicates restores mCherry open reading frame but the inserted sequence lacks three nucleotides compared to the intended insertion. See, FIG. 8F. These data demonstrate that a PEDAR system can repair genomic coding regions that are disrupted by large insertions.

### C. PEDAR System Therapeutic Applications

To test the clinical gene therapy embodiments of PEDAR systems, a Tyrosinemia I mouse model was selected, referred to as Fah^{ΔExon5}. This Tyrosinemia I mouse model is derived by replacing a 19-bp sequence with a ˜1.3-kb neo expression cassette at exon 5 of the Fah gene^{33,34}. See, FIG. 9A. This insertion disrupts the Fah gene to cause FAH protein deficiency and liver damage.

To maintain body weight and survival, Fah^{ΔExon5} mice are given water supplemented with NTBC (2-(2-nitro-4-trifluoromethylbenzoyl)-1,3-cyclohexanedione), a tyrosine catabolic pathway inhibitor. A PEDAR system was tested to correct a causative Fah^{ΔExon5} mutation by deleting a large mutation insertion and simultaneously inserting a 19-bp sequence back to repair exon 5. See, FIG. 9D. Two pegRNAs were engineered to target a genomic region before and after the inserted neo expression cassette, respectively. pegRNAs were designed comprising 3′ ends conjugated to a 22-bp RT template encoding an insertion nucleotide sequence (19 bp) plus a 3-bp sequence that was unintentionally deleted during the PECas9 deletion step. PECas9 and the two pegRNAs were delivered to the livers of Fah^{ΔExon5} mice (n=4) via hydrodynamic injection. Mice (n=2) treated with conventional Cas9 and pegRNAs served as a negative control. Mice were kept on water supplemented with NTBC after treatment. One week later, immunochemical staining was performed on liver sections with FAH antibody. FAH-expressing hepatocytes were detected on PECas9-treated liver sections, with a 0.76±0.25% correction rate. See, FIG. 9C and FIG. 9D, respectively. FAH protein expression was not detected in a conventional Cas9-treated mouse liver. See, FIG. 9C.

It has been reported that gene edited hepatocytes with a corrected FAH protein will gain a growth advantage and eventually repopulate the liver³⁵. Therefore, a PECas9 and two pegRNAs were delivered via hydrodynamic injection to Fah^{ΔExon5} mice (n=4) which were subsequently removed from the NTBC water supplement to allow repopulation of the gene edited hepatocytes. Untreated Fah^{ΔExon5} mice, but with removed NTBC water supplementation, were used as controls. Forty days later, widespread FAH protein patches were observed in PECas9-treated mouse liver sections, and the gene edited hepatocytes showed normal morphology. See, FIG. 9E and FIG. 10A.

To understand gene editing events in mouse liver, the insertion nucleotide sequence was amplified by using PCR primers spanning exon 5. A ˜300-bp deletion amplicon was identified in treated mice, indicating deletion of the ˜1.3-kb mutation insertion fragment. See, FIG. 9F. Deep sequencing of the ˜300-bp deletion amplicon showed an accurate deletion/insertion frequency of 78.2±³0.17% for total deletion events. See, FIG. 9G. Although it is not necessary to understand the mechanism of an invention, it is believed that hepatocytes with a gene-edited and corrected FAH protein will outgrow cells with unintended editing, thereby imposing a positive selection for cells with the edited gene. The average indel rates caused by each pegRNA at the Fah locus were 9.6% at cut site_F and 0.14% at cut site_R. See, FIG. 10B. It is noted that, even though Mouse 1 had a much higher average indel rate (27.7%) at cut site_F, it did not negatively affect FAH protein expression. See, FIG. 10B and FIG. 9E, respectively. Overall, these data demonstrate that PEDAR systems are gene therapy platforms which perform in vivo and repair pathogenic mutations.

In one embodiment, the present invention contemplates a Cas9 prime editor that operates on a PECas9-based deletion and repair (PEDAR) method that can correct mutations caused by large genomic rearrangements. Based on the design of conventional prime editors, the PEDAR system was modified to comprise a catalytically active Cas9 nuclease combined with an RT and paired pegRNAs. In operation, PECas9 couples together the replacement of a deletion nucleotide sequence with an insertion nucleotide sequence to accomplish a desired genome edit.

The presently disclosed PEDAR system is similar to a recently developed paired prime editing method, called PRIME-Del.³⁶ PRIME-Del, however, utilizes a Cas9 nickase protein (PE2) as opposed to a fully catalytically active Cas9 as in the PEDAR system. As such, unlike the PEDAR system, PRIME-Del is incapable of creating two DSBs for excising and replacing a large deletion sequence in excess of 1-10 kb with an insertion sequence. This difference in catalytic activity confers a distinct advantage of the PEDAR system over PRIME-Del, as the PEDAR system can create >10-kb target deletions simultaneously with up to 60-bp insertions in cells. PRIME-Del can only create 20- to 700-bp target deletions and up to 30-bp insertions. Consequently, the large sequence deletion/insertion capability of the PEDAR system is beyond the capability of either PRIME-Del or other conventional primer editors.^{17,36} Compared to PRIME-Del, PEDAR seems to be more error-prone, introducing higher fractions of direct deletion and imperfect deletion-insertion. See, FIG. 4G. However, both PRIME-Del and PEDAR exhibit comparable absolute accuracy rates in total genomic DNA. See, FIG. 411. Moreover, the PEDAR system performs deletion/insertion editing in quiescent hepatocytes in mouse liver, where HDR is not favorable³⁷. Thus, the PEDAR system is a robust genome editing technique to couple together larger nucleotide sequence deletions with a desired insertion sequence both in vitro and in vivo, than any other known prime editor system.

Despite the relative editing efficiency and accuracy of PECas9 being higher than conventional PE and conventional Cas9 gene editing, PECas9 activity can be further improved using multiple pegRNA sequences with distinct spacer sequences, PBSs, or RT templates. Furthermore, MMEJ or SSA enhancers could further improve the efficiency of PEDAR editing.^{38, 39}

### D. Implications of PEDAR In Correcting Genomic Duplications

In one embodiment, the present invention contemplates a PEDAR system for correcting genome duplications. See, FIG. 11A. Genome duplications have been reported to constitute ˜10% of all human pathogenic mutations, according to the ClinVar database¹.

One such genome duplication of high clinical significance is the trinucleotide CAG repeat expansion in the HTT gene, believed to result in Huntington disease⁴³. In one embodiment, the present invention contemplates a method comprising a PEDAR system that accurately removes an HTT gene CAG repeat expansion to reduce CAG repeat length and reduce the symptoms of Huntington disease.

Thus, the PEDAR system is a clinical platform for gene therapy. The significance of PEDAR also extends to basic biology, where it could be used for protein function studies. See, FIG. 11B. Previous studies have reported the introduction of in-frame deletions by a “tiling CRISPR” method to explore the functional domain of specific genomic coding or long non-coding regions^{44,45}. However, the PEDAR system exhibits a higher efficiency in mediating in-frame deletion compared to the canonical CRISPR/Cas9 system and would provide great advantages and superior data in comparison with the conventional tiling CRISPR methods.

### Experimental

### Example I

### Cell Culture and Transfection

Human embryonic kidney (HEK293T) cells (ATCC) and HEK293T-TLR cells24, 25 were maintained in Dulbecco's Modified Eagle's Medium (Corning) supplemented with 10% fetal bovine serum (Gibco) and 1% Penicillin/Streptomycin (Gibco).^{28,29} Cells were seeded at 70% confluence in 12-well cell culture plate one day before transfection. 1.5 μg PE-Cas9, and 1 μg paired pegRNAs (0.5 μg each) was transfected with Lipofectamine 3000 reagent (Invitrogen).

### Example II

### pegRNA Design and Clone

Plasmids expressing pegRNAs were constructed by Gibson assembly using BsaI-digested acceptor plasmid (Addgene #132777) as vector. See, Table 2.

TABLE 2
Sequences for pegRNAs

| | Spacer | |
|---|---|---|
| | Sequence | 3′ Extension Sequence |
| 979bp_pegRN | ggcccagactg | attaccctgttatccctacgtgctcagtctg |
| A_1 | agcacgtga | |
| 979bp_peg | gtgatcacctgc | tagggataacagggtaatatttgggcaggtg |
| RNA_2 | ccaaatgtg | |
| mCherry_peg | gtcgatcctcga | ttgctcaccatggtggcgctcgagga |
| RNA_1 | gcgccacca | |
| mCherry_peg | gcctcctcgccc | gcgccaccatggt gagcaagggcgag |
| RNA_2 | ttgctcaca | |
| Fah_peg | gacacggactt | cattggtggcatgctgccgagaagagtagaagtcc |
| RNA_1 | ctactcttct | |
| Fah_peg | gtctgaacataa | tctcggcagcatgccaccaatg ttggcattatgtt |
| RNA_2 | tgccaacat | |
| HEK3_CTT_i | ggcccagactg | tctgccatcaaagcgtgctcagtctg |
| ns_pegRNA | agcacgtga | |
| HEK3_pegR | ggcccagactg | ATTACCCTGTTATCCCTA cgtgctcagtctg |
| NA_F | agcacgtga | |
| HEK3_pegR | gtgatcacctgc | TAGGGATAACAGGGTAATatttgggcaggtg |
| NA_R | ccaaatgtg | |
| HEK3_pegR | ggcccagactg | tcacctgcccaaat ATTACCCTGTTATCCCTA cgtgctcagtctg |
| NA_alt_F | agcacgtga | |
| HEK3_pegR | gtgatcacctgc | ccagactgagcacgTAGGGATAACAGGGTAAT atttgggcaggtg |
| NA_alt_R | ccaaatgtg | |
| HEK3_10pbs | ggcccagactg | ATTACCCTGTTATCCCTAcgtgctcagt |
| pegRNA_F | agcacgtga | |
| HEK3_10pbs | gtgatcacctgc | TAGGGATAACAGGGTAATatttgggcag |
| pegRNA_R | ccaaatgtg | |
| HEK3_25pbs | ggcccagactg | ATTACCCTGTTATCCCTAcgtgctcagtctgggccccaaggat |
| pegRNA_F | agcacgtga | |
| HEK3_25pbs | gtgatcacctgc | TAGGGATAACAGGGTAATatttgggcaggtgatcaatgcttag |
| pegRNA_R | ccaaatgtg | |
| HEK3_44nt- | ggcccagactg | CACTTATCGT |
| ins | agcacgtga | CGTCATCCTTGTAATCATTACCCTGTTATCCCTAcgtgctc |
| pegRNA_F | | agtctg |
| HEK3_44nt- | gtgatcacctgc | TAGGGATAACAGGGTAATGATTACAAGGATGACGAC |
| ins | ccaaatgtg | GATAAGTGatttgggcaggtg |
| pegRNA_R | | |
| HEK3_60nt- | ggcccagactg | CAATAACTTC |
| ins | agcacgtga | GTATAATGTATGCTATACGAAGTTATAACAAT |
| pegRNA_F | | ATTACCCTGTTATCCCTAcgtgctcagtctg |
| HEK3_60nt- | gtgatcacctgc | TAGGGATAACAGGGTAATATTGTTATAACTTCGTATA |
| ins | ccaaatgtg | GCATACATTATACGAAGTTATTGatttgggcaggtg |
| pegRNA_R | | |
| DYRK1_peg | gtgtcaaatgat | ATTACCCTGTTATCCCTAgtttgtatcatttga |
| RNA_F | acaaacatt | |
| DYRK1_peg | gaaaagaccta | TAGGGATAACAGGGTAATttttgtttaggtc |
| RNA_R | aacaaaagaa | |
| CDC42_pegR | gcacaacaaac | ATTACCCTGTTATCCCTAgaaatttgtttgt |
| NA_F | aaatttccat | |
| CDC42_8k_ | gactagaaatac | TAGGGATAACAGGGTAATacagatgtatttc |
| pegRNA_R | atctgtttg | |
| CDC42_10k | gcttttgggttga | TAGGGATAACAGGGTAATgaaactcaaccca |
| pegRNA_R | gtttccgg | |
| mCherry_peg | gtcgatcctcga | ttgctcaccatggtggcgctcgagga |
| RNA_F | gcgccacca | |
| mCherry_peg | gcctcctcgccc | gcgccaccatggt gagcaagggcgag |
| RNA_R | ttgctcaca | |
| mCherry_peg | gtcctcctcgcc | gcgccaccatggtg agcaagggcgagg |
| RNA_R2 | cttgctcac | |
| Fah_peg | gacacggactt | cattggtggcatgctgccgagaagagtagaagtcc |
| RNA_1 | ctactcttct | |
| Fah_peg | gtctgaacataa | tctcggcagcatgccaccaatg ttggcattatgtt |
| RNA_2 | tgccaacat | |

### Example III

### Mouse Experiments

All animal study protocols were approved by the UMass Medical School IACUC. Fah^{ΔExon5} mice were kept on 10 mg/L NTBC water. Grompe et al., “Loss of fumarylacetoacetate hydrolase is responsible for the neonatal hepatic dysfunction phenotype of lethal albino mice” Genes & Development 7:2298-2307 (1993).

30 μg PE-Cas9 or Cas9 plasmid and 15 μg paired pegRNA expressing plasmids were 10 injected into 9-week-old mice. One week later, NTBC supplemented water was replaced with normal water, and mouse weight was measured every two days. When the mouse lost 20% of its body weight relative to the first day of measurement (day when NTBC water was removed), mouse will be supplemented with NTBC water until the body weight is back to original body weight. After 40 days, mice were euthanized.

### Example IV

### Immunohistochemistry

Portion of livers were fixed with 4% formalin, embedded in paraffin, sectioned at 5 m and stained with hematoxylin and eosin (H&E) for pathology. Liver sections were de-waxed, rehydrated, and stained using standard immunohistochemistry protocols. Xue et al., “Response and resistance to NF-kappaB inhibitors in mouse models of lung adenocarcinoma” Cancer Discovery 1:236-247 (2011).

The following antibody was used: anti-FAH (Abcam, 1:400). The images were captured using Leica DMi8 microscopy.

### Example V

### Genomic DNA Extraction, Amplification and Digestion

To extract genomic DNA, HEK293T cells (3 days post transfection) were washed with PBS, pelleted, and lysed with 50 μl Quick extraction buffer (Epicenter) and incubated in a 15 thermocycler (65° C. 15 min, and 98° C. 5 min). PureLink Genomic DNA Mini Kit (Thermo Fisher) was used to extract genomic DNA from two different liver lobes (˜10 mg each) per mouse.

Target sequences were amplified using Phusion Flash PCR Master Mix (Thermo Fisher) with the primers listed in Table 3.

TABLE 3
Primers

| | |
|---|---|
| 979bp_PCR_F | TAAGCAAGGGCTGATGTGGG |
| 979bp_PCR_R | tgtggtgccttttcggctta |
| mCherry_PCR_F | atgaccctgcgccttatttg |
| mCherry_PCR_R | TTGGTCACCTTCAGCTTGG |
| Fah_PCR_F | ggggttcctccatctaggtc |
| Fah_PCR_R | atgctgagggaaccaaaagc |
| 979bp_seqF | CTACACGACGCTCTTCCGATCT GCTGCAAGTAAGCATGCATTTG |
| 979bp_seqR | AGACGTGTGCTCTTCCGATCTtaggcgactgtccctctcaa |
| 979bp_indel1_seqF | CTACACGACGCTCTTCCGATCT GCTGCAAGTAAGCATGCATTTG |
| 979bp_indel1_seqR | AGACGTGTGCTCTTCCGATCTgctgcacatactagcccctg |
| 979bp_indel2_seqF | CTACACGACGCTCTTCCGATCTGACATTGCCATGCCAGCTAAG |
| 979bp_indel2_seqR | AGACGTGTGCTCTTCCGATCTtaggcgactgtccctctcaa |
| mCherry_seqF | CTACACGACGCTCTTCCGATCT AAGAGCTCACAACCCCTCAC |
| mCherry_seqR | AGACGTGTGCTCTTCCGATCTctcgccctcgatctcgaac |
| mCherry_indel1_seqR | CTACACGACGCTCTTCCGATCT AAGAGCTCACAACCCCTCAC |
| mCherry_indel1_seqF | AGACGTGTGCTCTTCCGATCTGATTTGTCTCGCCAAAGCCG |
| mCherry_indel1_seqR | CTACACGACGCTCTTCCGATCT AAGAGCTCACAACCCCTCAC |
| mCherry_indel2_seqF | CTACACGACGCTCTTCCGATCT TCGGCATGGACGAGCTGTA |
| mCherry_indel2_seqR | AGACGTGTGCTCTTCCGATCTctcgccctcgatctcgaac |
| Fah_seqF | CTACACGACGCTCTTCCGATCTCTGTTTGGGGTGTTCCCTCTG |
| Fah_seqR | AGACGTGTGCTCTTCCGATCTAAACAGGGTCTTTGCTGCTGG |
| Fah_indel1_seqF | CTACACGACGCTCTTCCGATCTGGGGTTCCTCCATCTAGGTCA |
| Fah_indel1_seqR | AGACGTGTGCTCTTCCGATCTATAGATTCGCCCTTGTGTCCC |
| Fah_indel2_seqF | CTACACGACGCTCTTCCGATCTGGATGCGGTGGGCTCTATG |
| Fah_indel2_seqR | AGACGTGTGCTCTTCCGATCTCCAGCATCTGGTCTAGGACATAC |
| HEK3_PCR_F | TAAGCAAGGGCTGATGTGGG |
| HEK3_PCR_R | tgtggtgccttttcggctta |
| DYRK1_PCR_F | GGTTTCACCTGGTTTGGGGA |
| DYRK1_PCR_F | AACAAGACACCAGGAAAAGACA |
| CDC42_8k_PCR_F | TTGCTCTGAGTGCCTGAACC |
| CDC42_8k_PCR_R | AGATGATCTTCTTAGGGCAAGAGT |
| CDC42_10k_PCR_F | GTGCCTGAACCTGTTGCTAAG |
| CDC42_10k_PCR_R | GAGGTTGCTCTAAGGTGGTGA |
| HEK3_indelF_seqF | TAAGCAAGGGCTGATGTGGG |
| HEK3_indelF_seqR | tgttgagctcgaccctgaag |
| HEK3_indelR_seqF | gacattgccatgccagctaag |
| HEK3_indelR_seqR | tgtggtgccttttcggctta |
| mCherry_PCR_F | atgaccctgcgccttatttg |
| mCherry_PCR_R | TTGGTCACCTTCAGCTTGG |
| Fah_PCR_F | ggggttcctccatctaggtc |
| Fah_PCR_R | atgctgagggaaccaaaagc |
| mCherry_indelR_seqF | CATGGTCCTGCTGGAGTTCGTG |
| mCherry_indelR_seqR | TTGGTCACCTTCAGCTTGG |
| HEK3_FL_qPCR_F | AAGTGGCCTTCTAGAGCTGG |
| HEK3_FL_qPCR_R | CTAGCTAGAGTGCTTGGGGC |
| HEK3_del_qPCR_F | CTGAGCACGTAGGGATAACAGG |
| HEK3_del_qPCR_R | TCAAATCCTCGCATTTGGGC |
| HEK3_seqF | CTACACGACGCTCTTCCGATCT GCTGCAAGTAAGCATGCATTTG |
| HEK3_seqR | AGACGTGTGCTCTTCCGATCTtaggcgactgtccctctcaa |
| DYRK1_seqF | CTACACGACGCTCTTCCGATCTACTGTTGTGTTGAGTAACATATACC |
| DYRK1_seqR | AGACGTGTGCTCTTCCGATCTAACAAGACACCAGGAAAAGACA |
| CDC42_8k_seqF | CTACACGACGCTCTTCCGATCT CAATTAAGTGTGTTGTTGTGGGC |
| CDC42_8k_seqR | AGACGTGTGCTCTTCCGATCTAGTATCTGATCAGCTTACCTTTTCT |
| CDC42_10k_seqF | CTACACGACGCTCTTCCGATCTTTAAGTGTGTTGTTGTGGGCG |
| CDC42_10k_seqR | AGACGTGTGCTCTTCCGATCT CTACAGTAGTGGGACAGGAAGC |
| mCherry_seqF | CTACACGACGCTCTTCCGATCT AAGAGCTCACAACCCCTCAC |
| mCherry_seqR | AGACGTGTGCTCTTCCGATCTctcgccctcgatctcgaac |
| Fah_seqF | CTACACGACGCTCTTCCGATCTCTGTTTGGGGTGTTCCCTCTG |
| Fah_seqR | AGACGTGTGCTCTTCCGATCTAAACAGGGTCTTTGCTGCTGG |
| Fah_indell_seqF | CTACACGACGCTCTTCCGATCTGGGGTTCCTCCATCTAGGTCA |
| Fah_indell_seqR | AGACGTGTGCTCTTCCGATCTATAGATTCGCCCTTGTGTCCC |
| Fah_indel2_seqF | CTACACGACGCTCTTCCGATCTGGATGCGGTGGGCTCTATG |
| Fah_indel2_seqR | AGACGTGTGCTCTTCCGATCTCCAGCATCTGGTCTAGGACATAC |

PCR products were analyzed by electrophoresis in a 1% agarose gel, and target amplicons were extracted using DNA extraction kit (Qiagen).

10 ng of purified PCR products were incubated with I-SceI endonuclease (NEB) according to manufacture's instruction. One-hour post incubation, the product was visualized and analyzed by electrophoresis in 4-20% TBE gel (Thermo).

### Example VI

### Tracking of Indels by Decomposition (TIDE) Analysis

The sequences around the two cut sites of the target locus were amplified using Phusion Flash PCR Master Mix (Thermo Fisher) with the primers as listed in Table 2 (supra). Sanger sequencing was performed to sequence the purified PCR products, and the trace sequences were analyzed using TIDE software (tide.nki.nl). The alignment window of left boundary was set at 10-bp.

### Example VII

### Quantification of Total Genomic DNA

Real-time quantitative PCR (qPCR) was used to calculate the absolute editing rate in total genomic DNA at the HEK3 locus. Quantitative PCR was performed with SsoFast EvaGreen Supermix (Bio-rad). Primers within the deletion region (P1 and P2), spanning the deletion region (P3 and P4), or across the deletion/insertion junction (P5 and P6) were designed. See, FIG. 12A. Two 250-bp DNA fragments (referred to as WT and Edited) of the same sequence with unedited or accurately edited target site were designed and serially diluted, serving as standard templates. See, FIG. 12B. Using indicated primers and templates to perform quantitative PCR, three standard curves were generated, reflecting the correlation between qPCR cycle number and the concentration of DNA without 991-bp deletion, with 991-bp deletion, or with accurate 991-bp deletion/18-bp insertion. See, FIGS. 12C, 12D & 12E, respectively. Finally, three rounds of quantitative PCR were performed using the edited genomic DNA as template and corresponding primer pairs (P1+P2, P3+P4, or P5+P6). The standard curves were applied to calculate the absolute copy number of genomic DNA with deletion, without deletion, or with accurate deletion-insertion.

The absolute rates of each type of editing introduced by PEDAR were calculated as follows: (1) Accurate deletion-insertion editing rate=copy number of DNA with accurate deletion-insertion/copy number of DNA with and without deletion. (2) Other deletion-insertion rate=(copy number of DNA with deletion—copy number of DNA with accurate deletion-insertion)/copy number of DNA with and without deletion. (3) Absolute rate of small indels at two cut sites=copy number of DNA without deletion x indel rate at distinct cut site calculated by TIDE/copy number of DNA with and without deletion.

### Example VII

### Flow Cytometry Analysis

To assess mCherry recovery rate, post-editing HEK293T-TLR cells were trypsinized and analyzed using the MACSQuant VYB Flow Cytometer. Untreated HEK293T-TLR cells were used as a negative control for gating. All data were analyzed by FlowJo10.0 software.

### Example IX

### High Throughput DNA Sequencing of Genomic DNA Samples

Genomic sites of interest were amplified from genomic DNA using specific primers containing llumina forward and reverse adaptors. See, Table 2. To quantify the percentage of target deletion-insertion by PE-Cas9 or Cas9, an amplification was performed on the fragment containing deletions (˜200 bp in length) from total genomic DNA to exclude length-dependent bias during PCR amplification.

20 μL PCR1 reactions were performed with 0.5 μM each of forward and reverse primer, 1 μL of genomic DNA extract or 300 ng purified genomic DNA, and 10 μL of Phusion Flash PCR Master Mix (Thermo Fisher). PCR reactions were carried out as follows: 98° C. for 10 s, then 20 cycles of [98° C. for 1 s, 55° C. for 5 s, and 72° C. for 10 s], followed by a final 72° C. extension for 3 min.

After the first round of PCR, unique Illumina barcoding reverse primer was added to each sample in a secondary PCR reaction (PCR 2). Specifically, 20 μL of a PCR reaction contained 0.5 μM of unique reverse Illumina barcoding primer pair and 0.5 μM common forward Illumina barcoding primer, 1 μL of unpurified PCR 1 reaction mixture, and 10 μL of Phusion Flash PCR Master Mix. The barcoding PCR2 reactions were carried out as follows: 98° C. for 10 s, then 20 cycles of [98° C. for 1 s, 60° C. for 5 s, and 72° C. for 10 s], followed by a final 72° C. extension for 3 min. PCR 2 products were purified by 1% agarose gel using a QIAquick Gel Extraction Kit (Qiagen), eluting with 15 μL of Elution Buffer.

DNA concentration was measured by Bioanalyzer and sequenced on an Illumina MiSeq instrument (150 bp, paired-end) according to the manufacturer's protocols. Paired-end reads were merged with FLASh41 with maximum overlap length equal to 150 bp. Alignment of amplicon sequence to the reference sequence was performed using CRISPResso242.

To quantify accurate deletion-insertion edits, CRISPResso2 was run in HDR mode using the sequence with desired deletion/insertion editing as the reference sequence. The editing window is set to 10 bp. Editing yield was calculated as: [# of HDR aligned reads]+[total reads]. For all experiments, indel yields were calculated as: [# of indel-containing reads]+[total reads].

### Example X

### ClinVar Data Analysis

The ClinVar variant summary was obtained from NCBI ClinVar database (accessed Dec. 31, 2020). Variants with pathogenic significance were filtered by allele ID to remove duplicates. All pathogenic variants were categorized according to mutation type. The fractions of distinct mutation types were calculated using GraphPad Prism8.

### REFERENCES

1. Landrum, M. J. et al. ClinVar: public archive of relationships among sequence variation and human phenotype. Nucleic Acids Res 42, D980-985 (2014).
2. Cordaux, R. & Batzer, M. A. The impact of retrotransposons on human genome evolution. Nat Rev Genet 10, 691-703 (2009).
3. Chen, J. M., Stenson, P. D., Cooper, D. N. & Ferec, C. A systematic analysis of LINE-1 endonuclease-dependent retrotranspositional events causing human genetic disease. Hum Genet 117, 411-427 (2005).
4. Hancks, D. C. & Kazazian, H. H. Roles for retrotransposon insertions in human disease. Mobile DNA 7, 9 (2016).
5. Wang, L., Norris, E. T. & Jordan, I. K. Human Retrotransposon Insertion Polymorphisms Are Associated with Health and Disease via Gene Regulatory Phenotypes. Front Microbiol 8, 1418 (2017).
6. Hancks, D. C. & Kazazian, H. H., Jr. Active human retrotransposons: variation and disease. Curr Opin Genet Dev 22, 191-203 (2012).
7. Qian, Y. et al. Identification of pathogenic retrotransposon insertions in cancer predisposition genes. Cancer Genet 216-217, 159-169 (2017).
8. Ran, F. A. et al. Double nicking by RNA-guided CRISPR Cas9 for enhanced genome editing specificity. Cell 154, 1380-1389 (2013).
9. Cong, L. et al. Multiplex genome engineering using CRISPR/Cas systems. Science 339, 819-823 (2013).
10. Kato, T. et al. Creation of mutant mice with megabase-sized deletions containing custom-designed breakpoints by means of the CRISPR/Cas9 system. Sci Rep 7, 59 (2017).
11. Hara, S. et al. Microinjection-based generation of mutant mice with a double mutation and a 0.5 Mb deletion in their genome by the CRISPR/Cas9 system. J Reprod Dev 62, 531-536 (2016).
12. Wang, L. et al. Large genomic fragment deletion and functional gene cassette knock-in via Cas9 protein mediated genome editing in one-cell rodent embryos. Sci Rep 5, 17517 (2015).
13. Yeh, C. D., Richardson, C. D. & Corn, J. E. Advances in genome editing through control of DNA repair pathways. Nat Cell Biol 21, 1468-1478 (2019).
14. Zheng, Q. et al. Precise gene deletion and replacement using the CRISPR/Cas9 system in human cells. Biotechniques 57, 115-124 (2014).
15. Cox, D. B., Platt, R. J. & Zhang, F. Therapeutic genome editing: prospects and challenges. Nature medicine 21, 121-131 (2015).
16. Liu, M. et al. Methodologies for Improving HDR Efficiency. Front Genet 9, 691 (2018).
17. Anzalone, A. V. et al. Search-and-replace genome editing without double-strand breaks or donor DNA. Nature 576, 149-157 (2019).
18. Matsoukas, I. G. Prime Editing: Genome Editing for Rare Genetic Diseases Without Double-Strand Breaks or Donor DNA. Front Genet 11, 528 (2020).
19. Liu, P. et al. Improved prime editors enable pathogenic allele correction and cancer modelling in adult mice. Nat Commun 12, 2121 (2021).
20. Jang, H. et al. Prime editing enables precise genome editing in mouse liver and retina. bioRxiv, 2021.2001.2008.425835 (2021).
21. Schene, I. F. et al. Prime editing for functional repair in patient-derived disease models. Nat Commun 11, 5352 (2020).
22. Jiang, Y. Y. et al. Prime editing efficiently generates W542L and S621I double mutations in two ALS genes in maize. Genome biology 21, 257 (2020).
23. Song, X., Huang, H., Xiong, Z., Ai, L. & Yang, S. CRISPR-Cas9(D10A) Nickase-Assisted Genome Editing in Lactobacillus casei. Appl Environ Microbiol 83 (2017).
24. Cho, S. W. et al. Analysis of off-target effects of CRISPR/Cas-derived RNA-guided endonucleases and nickases. Genome Res 24, 132-141 (2014).
25. Sfeir, A. & Symington, L. S. Microhomology-Mediated End Joining: A Back-up Survival Mechanism or Dedicated Pathway? Trends Biochem Sci 40, 701-714 (2015).
26. Bhargava, R., Onyango, D. O. & Stark, J. M. Regulation of Single-Strand Annealing and its Role in Genome Maintenance. Trends Genet 32, 566-575 (2016).
27. Kim, H. K. et al. Predicting the efficiency of prime editing guide RNAs in human cells. Nat Biotechnol 39, 198-206 (2021).
28. Mir, A. et al. Heavily and fully modified RNAs guide efficient SpyCas9-mediated genome editing. Nat Commun 9, 2641 (2018).
29. Certo, M. T. et al. Tracking genome engineering outcome at individual DNA breakpoints. Nat Methods 8, 671-676 (2011).
30. Zhan, H., Li, A., Cai, Z., Huang, W. & Liu, Y. Improving transgene expression and CRISPR-Cas9 efficiency with molecular engineering-based molecules. Clin Transl Med 10, e194 (2020).
31. Chen, R. et al. Enrichment of transiently transfected mesangial cells by cell sorting after cotransfection with GFP. Am J Physiol 276, F777-785 (1999).
32. Homann, S. et al. A novel rapid and reproducible flow cytometric method for optimization of transfection efficiency in cells. PloS one 12, e0182941 (2017).
33. Pham, C. T., MacIvor, D. M., Hug, B. A., Heusel, J. W. & Ley, T. J. Long-range disruption of gene expression by a selectable marker cassette. Proceedings of the National Academy of Sciences of the United States of America 93, 13090-13095 (1996).
34. Grompe, M. et al. Loss of fumarylacetoacetate hydrolase is responsible for the neonatal hepatic dysfunction phenotype of lethal albino mice. Genes & development 7, 2298-2307 (1993).
35. Paulk, N. K. et al. Adeno-associated virus gene repair corrects a mouse model of hereditary tyrosinemia in vivo. Hepatology 51, 1200-1208 (2010).
36. Choi, J. et al. Precise genomic deletions using paired prime editing. bioRxiv, 2020.2012.2030.424891 (2021).
37. VanLith, C. J. et al. Ex Vivo Hepatocyte Reprograming Promotes Homology-Directed DNA Repair to Correct Metabolic Disease in Mice After Transplantation. Hepatol Commun 3, 558-573 (2019).
38. Aida, T. et al. Gene cassette knock-in in mammalian cells and zygotes by enhanced MMEJ. BMC Genomics 17, 979 (2016).
39. Dutta, A. et al. Microhomology-mediated end joining is activated in irradiated human cells due to phosphorylation-dependent formation of the XRCC1 repair complex. Nucleic Acids Research 45, 2585-2599 (2016).
40. Kim, D. Y., Moon, S. B., Ko, J. H., Kim, Y. S. & Kim, D. Unbiased investigation of specificities of prime editing systems in human cells. Nucleic Acids Res 48, 10576-10589 (2020).
41. Jin, S. et al. Genome-wide specificity of prime editors in plants. Nat Biotechnol (2021).
42. Naeem, M., Majeed, S., Hoque, M. Z. & Ahmad, I. Latest Developed Strategies to Minimize the Off-Target Effects in CRISPR-Cas-Mediated Genome Editing. Cells 9 (2020).
43. Warby, S. C. et al. CAG expansion in the Huntington disease gene is associated with a specific and targetable predisposing haplogroup. Am J Hum Genet 84, 351-366 (2009).
44. Wang, Y. et al. Identification of a Xist silencing domain by Tiling CRISPR. Sci Rep 9, 2408 (2019).
45. He, W. et al. De novo identification of essential protein domains from CRISPR-Cas9 tiling-sgRNA knockout screens. Nat Commun 10, 4541 (2019).
46. Xue, W. et al. Response and resistance to NF-kappaB inhibitors in mouse models of lung adenocarcinoma. Cancer discovery 1, 236-247 (2011).
47. Magoc, T. & Salzberg, S. L. FLASH: fast length adjustment of short reads to improve genome assemblies. Bioinformatics 27, 2957-2963 (2011).
48. Clement, K. et al. CRISPResso2 provides accurate and rapid genome editing sequence analysis. Nat Biotechnol 37, 224-226 (2019).

We claim:

1. A method, comprising:
a) providing;
i) a genomic DNA locus comprising a target nucleotide sequence; and
ii) a composition comprising a catalytically active Cas9 protein fused to a reverse transcriptase, a first prime editor guide RNA (pegRNA) molecule conjugated to a first reverse transcriptase DNA insertion template and a second prime editor guide RNA molecule conjugated to a second reverse transcriptase DNA insertion template, wherein said first and second reverse transcriptase DNA templates are complementary;
b) contacting said catalytically active Cas9 protein with said target nucleotide sequence, wherein said first pegRNA molecule binds to a sense strand of said target nucleotide sequence and said second pegRNA molecule binds to an antisense strand of said target nucleotide sequence;
c) creating two double strand breaks in said target nucleotide sequence with said catalytically active Cas9 protein such that said target nucleotide sequence is deleted; and
d) incorporating a double stranded insertion nucleotide sequence encoded by said first and second reverse transcriptase insertion templates into said genomic DNA sequence.

2. The method of claim 1, wherein said target nucleotide sequence ranges between 1 kb to 10 kb.

3. The method of claim 1, wherein said insertion nucleotide sequence has a length of up to 60 bp.

4. The method of claim 1, wherein said target nucleotide sequence is linked to a genetic disease.

5. The method of claim 4, wherein said genetic disease is tyrosinemia I.

6. The method of claim 4, said target nucleotide sequence comprises a Fah^{ΔExon5} mutation.

7. A method, comprising:
a) providing;
i) a patient exhibiting at least one symptom of a genetic disease; and
ii) a composition comprising a catalytically active Cas9 protein fused to a reverse transcriptase, a first prime editor guide RNA (pegRNA) molecule conjugated to a first reverse transcriptase DNA insertion template and a second prime editor guide RNA molecule conjugated to a second reverse transcriptase DNA insertion template, wherein said first and second reverse transcriptase DNA templates are complementary;
b) administering said composition to said patient such that said at least one symptom of said genetic disease is reduced.

8. The method of claim 1, wherein said genetic disease is tyrosinemia.

9. The method of claim 1, wherein said genetic disease is Huntington disease.

10. The method of claim 1, wherein said patient further comprises a gene mutation insertion between 1 kb-10 kb.

11. The method of claim 10, wherein said administering replaces said gene mutation insertion with an insertion nucleotide sequence that has a length of up to 60 bp.

12. A composition comprising a catalytically active Ca9 protein fused to a reverse transcriptase, a first prime editor guide RNA (pegRNA) molecule conjugated to a first reverse transcriptase DNA template and a second prime editor guide RNA molecule conjugated to a second reverse transcriptase DNA template, wherein said first and second reverse transcriptase DNA templates are complementary.

13. The composition of claim 12, wherein said first reverse transcriptase DNA template is conjugated as a 3′ extension to said first pegRNA molecule.

14. The composition of claim 12, wherein second reverse transcriptase DNA template is conjugated as a 3′ extension to the second pegRNA molecule.

15. The composition of claim 12, wherein said first and second reverse transcriptase DNA templates have a length of up to 60 bp.


SEQUENCE LISTING (162 sequences)

SEQ ID NO: 1 - DNA, 42 bp, Artificial Sequence
  Synthetic
ttggggcccagactgagcacgatttgggcaggtgatcaatgc

SEQ ID NO: 2 - DNA, 84 bp, Artificial Sequence
  Synthetic
ttggggcccagactgagcacgtagggataacagggtaatgcaccgactcggtcccactttttcatttgggcaggtgatcaatgc

SEQ ID NO: 3 - DNA, 82 bp, Artificial Sequence
  Synthetic
ttggggcccagactgagcacgtagggataacagggtaatgcaccgactcggtcccacttttatttgggcaggtgatcaatgc

SEQ ID NO: 4 - DNA, 68 bp, Artificial Sequence
  Synthetic
ttggggcccagactgagcacgtagggataacagggtaatgcaccgactcggtcccactttttcaatgc

SEQ ID NO: 5 - DNA, 89 bp, Artificial Sequence
  Synthetic
ttggggcccagactgagcacgtagggataacagggtaatgcaccgactcggtcccactttttcaagttatttgggcaggtgatcaatgc

SEQ ID NO: 6 - DNA, 41 bp, Artificial Sequence
  Synthetic
ttggggcccagactgagcacgtttgggcaggtgatcaatgc

SEQ ID NO: 7 - DNA, 61 bp, Artificial Sequence
  Synthetic
ttggggcccagactgagcacgctagggataacagggtaatatttgggcaggtgatcaatgc

SEQ ID NO: 8 - DNA, 57 bp, Artificial Sequence
  Synthetic
ttggggcccagactgagctagggataacagggtaatatttgggcaggtgatcaatgc

SEQ ID NO: 9 - DNA, 42 bp, Artificial Sequence; identical to SEQ ID NO: 1
  Synthetic

SEQ ID NO: 10 - DNA, 60 bp, Artificial Sequence
  Synthetic
ttggggcccagactgagcacgtagggataacagggtaatatttgggcaggtgatcaatgc

SEQ ID NO: 11 - DNA, 80 bp, Artificial Sequence
  Synthetic
ttggggcccagactgagcacgtagggataacagggtaatgctagggataacagggtaatatttgggcaggtgatcaatgc

SEQ ID NO: 12 - DNA, 41 bp, Artificial Sequence; identical to SEQ ID NO: 6
  Synthetic

SEQ ID NO: 13 - DNA, 57 bp, Artificial Sequence; identical to SEQ ID NO: 8
  Synthetic

SEQ ID NO: 14 - DNA, 63 bp, Artificial Sequence
  Synthetic
ttggggcccagactgagcacgtgctagggataacagggtaatatttgggcaggtgatcaatgc

SEQ ID NO: 15 - DNA, 41 bp, Artificial Sequence
  Synthetic
ttggggcccagactgagcacgtagggataacagggtaatgc

SEQ ID NO: 16 - DNA, 84 bp, Artificial Sequence; identical to SEQ ID NO: 2
  Synthetic

SEQ ID NO: 17 - DNA, 41 bp, Artificial Sequence
  Synthetic
ttggggcccagactgagcacatttgggcaggtgatcaatgc

SEQ ID NO: 18 - DNA, 60 bp, Artificial Sequence; identical to SEQ ID NO: 10
  Synthetic

SEQ ID NO: 19 - DNA, 42 bp, Artificial Sequence; identical to SEQ ID NO: 1
  Synthetic

SEQ ID NO: 20 - DNA, 80 bp, Artificial Sequence; identical to SEQ ID NO: 11
  Synthetic

SEQ ID NO: 21 - DNA, 57 bp, Artificial Sequence; identical to SEQ ID NO: 8
  Synthetic

SEQ ID NO: 22 - DNA, 88 bp, Artificial Sequence
  Synthetic
ttggggcccagactgagcacgtagggataacagggtaatgcaccgactctagggataacagggtaatatttgggcaggtgatcaatgc

SEQ ID NO: 23 - DNA, 84 bp, Artificial Sequence; identical to SEQ ID NO: 2
  Synthetic

SEQ ID NO: 24 - DNA, 63 bp, Artificial Sequence; identical to SEQ ID NO: 14
  Synthetic

SEQ ID NO: 25 - DNA, 83 bp, Artificial Sequence
  Synthetic
ttggggcccagactgagcacgtagggataacagggtaatgcaccgactcggtcccactttttatttgggcaggtgatcaatgc

SEQ ID NO: 26 - DNA, 61 bp, Artificial Sequence; identical to SEQ ID NO: 7
  Synthetic

SEQ ID NO: 27 - DNA, 20 bp, Artificial Sequence
  Synthetic
ggcccagactgagcacgtga

SEQ ID NO: 28 - DNA, 31 bp, Artificial Sequence
  Synthetic
attaccctgttatccctacgtgctcagtctg

SEQ ID NO: 29 - DNA, 21 bp, Artificial Sequence
  Synthetic
gtgatcacctgcccaaatgtg

SEQ ID NO: 30 - DNA, 31 bp, Artificial Sequence
  Synthetic
tagggataacagggtaatatttgggcaggtg

SEQ ID NO: 31 - DNA, 21 bp, Artificial Sequence
  Synthetic
gtcgatcctcgagcgccacca

SEQ ID NO: 32 - DNA, 26 bp, Artificial Sequence
  Synthetic
ttgctcaccatggtggcgctcgagga

SEQ ID NO: 33 - DNA, 21 bp, Artificial Sequence
  Synthetic
gcctcctcgcccttgctcaca

SEQ ID NO: 34 - DNA, 26 bp, Artificial Sequence
  Synthetic
gcgccaccatggtgagcaagggcgag

SEQ ID NO: 35 - DNA, 21 bp, Artificial Sequence
  Synthetic
gacacggacttctactcttct

SEQ ID NO: 36 - DNA, 35 bp, Artificial Sequence
  Synthetic
cattggtggcatgctgccgagaagagtagaagtcc

SEQ ID NO: 37 - DNA, 21 bp, Artificial Sequence
  Synthetic
gtctgaacataatgccaacat

SEQ ID NO: 38 - DNA, 35 bp, Artificial Sequence
  Synthetic
tctcggcagcatgccaccaatgttggcattatgtt

SEQ ID NO: 39 - DNA, 20 bp, Artificial Sequence
  Synthetic
ggcccagactgagcacgtga

SEQ ID NO: 40 - DNA, 26 bp, Artificial Sequence
  Synthetic
tctgccatcaaagcgtgctcagtctg

SEQ ID NO: 41 - DNA, 20 bp, Artificial Sequence
  Synthetic
ggcccagactgagcacgtga

SEQ ID NO: 42 - DNA, 31 bp, Artificial Sequence; identical to SEQ ID NO: 28
  Synthetic

SEQ ID NO: 43 - DNA, 21 bp, Artificial Sequence
  Synthetic
gtgatcacctgcccaaatgtg

SEQ ID NO: 44 - DNA, 31 bp, Artificial Sequence; identical to SEQ ID NO: 30
  Synthetic

SEQ ID NO: 45 - DNA, 20 bp, Artificial Sequence
  Synthetic
ggcccagactgagcacgtga

SEQ ID NO: 46 - DNA, 45 bp, Artificial Sequence
  Synthetic
tcacctgcccaaatattaccctgttatccctacgtgctcagtctg

SEQ ID NO: 47 - DNA, 21 bp, Artificial Sequence
  Synthetic
gtgatcacctgcccaaatgtg

SEQ ID NO: 48 - DNA, 45 bp, Artificial Sequence
  Synthetic
ccagactgagcacgtagggataacagggtaatatttgggcaggtg

SEQ ID NO: 49 - DNA, 20 bp, Artificial Sequence
  Synthetic
ggcccagactgagcacgtga

SEQ ID NO: 50 - DNA, 28 bp, Artificial Sequence
  Synthetic
attaccctgttatccctacgtgctcagt

SEQ ID NO: 51 - DNA, 21 bp, Artificial Sequence
  Synthetic
gtgatcacctgcccaaatgtg

SEQ ID NO: 52 - DNA, 28 bp, Artificial Sequence
  Synthetic
tagggataacagggtaatatttgggcag

SEQ ID NO: 53 - DNA, 20 bp, Artificial Sequence
  Synthetic
ggcccagactgagcacgtga

SEQ ID NO: 54 - DNA, 43 bp, Artificial Sequence
  Synthetic
attaccctgttatccctacgtgctcagtctgggccccaaggat

SEQ ID NO: 55 - DNA, 21 bp, Artificial Sequence
  Synthetic
gtgatcacctgcccaaatgtg

SEQ ID NO: 56 - DNA, 43 bp, Artificial Sequence
  Synthetic
tagggataacagggtaatatttgggcaggtgatcaatgcttag

SEQ ID NO: 57 - DNA, 20 bp, Artificial Sequence
  Synthetic
ggcccagactgagcacgtga

SEQ ID NO: 58 - DNA, 57 bp, Artificial Sequence
  Synthetic
cacttatcgtcgtcatccttgtaatcattaccctgttatccctacgtgctcagtctg

SEQ ID NO: 59 - DNA, 21 bp, Artificial Sequence
  Synthetic
gtgatcacctgcccaaatgtg

SEQ ID NO: 60 - DNA, 57 bp, Artificial Sequence
  Synthetic
tagggataacagggtaatgattacaaggatgacgacgataagtgatttgggcaggtg

SEQ ID NO: 61 - DNA, 20 bp, Artificial Sequence
  Synthetic
ggcccagactgagcacgtga

SEQ ID NO: 62 - DNA, 73 bp, Artificial Sequence
  Synthetic
caataacttcgtataatgtatgctatacgaagttataacaatattaccctgttatccctacgtgctcagtctg

SEQ ID NO: 63 - DNA, 21 bp, Artificial Sequence
  Synthetic
gtgatcacctgcccaaatgtg

SEQ ID NO: 64 - DNA, 73 bp, Artificial Sequence
  Synthetic
tagggataacagggtaatattgttataacttcgtatagcatacattatacgaagttattgatttgggcaggtg

SEQ ID NO: 65 - DNA, 21 bp, Artificial Sequence
  Synthetic
gtgtcaaatgatacaaacatt

SEQ ID NO: 66 - DNA, 33 bp, Artificial Sequence
  Synthetic
attaccctgttatccctagtttgtatcatttga

SEQ ID NO: 67 - DNA, 21 bp, Artificial Sequence
  Synthetic
gaaaagacctaaacaaaagaa

SEQ ID NO: 68 - DNA, 31 bp, Artificial Sequence
  Synthetic
tagggataacagggtaatttttgtttaggtc

SEQ ID NO: 69 - DNA, 21 bp, Artificial Sequence
  Synthetic
gcacaacaaacaaatttccat

SEQ ID NO: 70 - DNA, 31 bp, Artificial Sequence
  Synthetic
attaccctgttatccctagaaatttgtttgt

SEQ ID NO: 71 - DNA, 21 bp, Artificial Sequence
  Synthetic
gactagaaatacatctgtttg

SEQ ID NO: 72 - DNA, 31 bp, Artificial Sequence
  Synthetic
tagggataacagggtaatacagatgtatttc

SEQ ID NO: 73 - DNA, 21 bp, Artificial Sequence
  Synthetic
gcttttgggttgagtttccgg

SEQ ID NO: 74 - DNA, 31 bp, Artificial Sequence
  Synthetic
tagggataacagggtaatgaaactcaaccca

SEQ ID NO: 75 - DNA, 21 bp, Artificial Sequence
  Synthetic
gtcgatcctcgagcgccacca

SEQ ID NO: 76 - DNA, 26 bp, Artificial Sequence
  Synthetic
ttgctcaccatggtggcgctcgagga

SEQ ID NO: 77 - DNA, 21 bp, Artificial Sequence
  Synthetic
gcctcctcgcccttgctcaca

SEQ ID NO: 78 - DNA, 26 bp, Artificial Sequence
  Synthetic
gcgccaccatggtgagcaagggcgag

SEQ ID NO: 79 - DNA, 21 bp, Artificial Sequence
  Synthetic
gtcctcctcgcccttgctcac

SEQ ID NO: 80 - DNA, 27 bp, Artificial Sequence
  Synthetic
gcgccaccatggtgagcaagggcgagg

SEQ ID NO: 81 - DNA, 21 bp, Artificial Sequence
  Synthetic
gacacggacttctactcttct

SEQ ID NO: 82 - DNA, 35 bp, Artificial Sequence; identical to SEQ ID NO: 36
  Synthetic

SEQ ID NO: 83 - DNA, 21 bp, Artificial Sequence
  Synthetic
gtctgaacataatgccaacat

SEQ ID NO: 84 - DNA, 35 bp, Artificial Sequence; identical to SEQ ID NO: 38
  Synthetic

SEQ ID NO: 85 - DNA, 20 bp, Artificial Sequence
  Synthetic
taagcaagggctgatgtggg

SEQ ID NO: 86 - DNA, 20 bp, Artificial Sequence
  Synthetic
tgtggtgccttttcggctta

SEQ ID NO: 87 - DNA, 20 bp, Artificial Sequence
  Synthetic
atgaccctgcgccttatttg

SEQ ID NO: 88 - DNA, 19 bp, Artificial Sequence
  Synthetic
ttggtcaccttcagcttgg

SEQ ID NO: 89 - DNA, 20 bp, Artificial Sequence
  Synthetic
ggggttcctccatctaggtc

SEQ ID NO: 90 - DNA, 20 bp, Artificial Sequence
  Synthetic
atgctgagggaaccaaaagc

SEQ ID NO: 91 - DNA, 44 bp, Artificial Sequence
  Synthetic
ctacacgacgctcttccgatctgctgcaagtaagcatgcatttg

SEQ ID NO: 92 - DNA, 41 bp, Artificial Sequence
  Synthetic
agacgtgtgctcttccgatcttaggcgactgtccctctcaa

SEQ ID NO: 93 - DNA, 44 bp, Artificial Sequence; identical to SEQ ID NO: 91
  Synthetic

SEQ ID NO: 94 - DNA, 41 bp, Artificial Sequence
  Synthetic
agacgtgtgctcttccgatctgctgcacatactagcccctg

SEQ ID NO: 95 - DNA, 43 bp, Artificial Sequence
  Synthetic
ctacacgacgctcttccgatctgacattgccatgccagctaag

SEQ ID NO: 96 - DNA, 41 bp, Artificial Sequence; identical to SEQ ID NO: 92
  Synthetic

SEQ ID NO: 97 - DNA, 42 bp, Artificial Sequence
  Synthetic
ctacacgacgctcttccgatctaagagctcacaacccctcac

SEQ ID NO: 98 - DNA, 40 bp, Artificial Sequence
  Synthetic
agacgtgtgctcttccgatctctcgccctcgatctcgaac

SEQ ID NO: 99 - DNA, 42 bp, Artificial Sequence; identical to SEQ ID NO: 97
  Synthetic

SEQ ID NO: 100 - DNA, 41 bp, Artificial Sequence
  Synthetic
agacgtgtgctcttccgatctgatttgtctcgccaaagccg

SEQ ID NO: 101 - DNA, 41 bp, Artificial Sequence
  Synthetic
ctacacgacgctcttccgatcttcggcatggacgagctgta

SEQ ID NO: 102 - DNA, 40 bp, Artificial Sequence; identical to SEQ ID NO: 98
  Synthetic

SEQ ID NO: 103 - DNA, 43 bp, Artificial Sequence
  Synthetic
ctacacgacgctcttccgatctctgtttggggtgttccctctg

SEQ ID NO: 104 - DNA, 42 bp, Artificial Sequence
  Synthetic
agacgtgtgctcttccgatctaaacagggtctttgctgctgg

SEQ ID NO: 105 - DNA, 43 bp, Artificial Sequence
  Synthetic
ctacacgacgctcttccgatctggggttcctccatctaggtca

SEQ ID NO: 106 - DNA, 42 bp, Artificial Sequence
  Synthetic
agacgtgtgctcttccgatctatagattcgcccttgtgtccc

SEQ ID NO: 107 - DNA, 41 bp, Artificial Sequence
  Synthetic
ctacacgacgctcttccgatctggatgcggtgggctctatg

SEQ ID NO: 108 - DNA, 44 bp, Artificial Sequence
  Synthetic
agacgtgtgctcttccgatctccagcatctggtctaggacatac

SEQ ID NO: 109 - DNA, 20 bp, Artificial Sequence
  Synthetic
taagcaagggctgatgtggg

SEQ ID NO: 110 - DNA, 20 bp, Artificial Sequence
  Synthetic
tgtggtgccttttcggctta

SEQ ID NO: 111 - DNA, 20 bp, Artificial Sequence
  Synthetic
ggtttcacctggtttgggga

SEQ ID NO: 112 - DNA, 22 bp, Artificial Sequence
  Synthetic
aacaagacaccaggaaaagaca

SEQ ID NO: 113 - DNA, 20 bp, Artificial Sequence
  Synthetic
ttgctctgagtgcctgaacc

SEQ ID NO: 114 - DNA, 24 bp, Artificial Sequence
  Synthetic
agatgatcttcttagggcaagagt

SEQ ID NO: 115 - DNA, 21 bp, Artificial Sequence
  Synthetic
gtgcctgaacctgttgctaag

SEQ ID NO: 116 - DNA, 21 bp, Artificial Sequence
  Synthetic
gaggttgctctaaggtggtga

SEQ ID NO: 117 - DNA, 20 bp, Artificial Sequence
  Synthetic
taagcaagggctgatgtggg

SEQ ID NO: 118 - DNA, 20 bp, Artificial Sequence
  Synthetic
tgttgagctcgaccctgaag

SEQ ID NO: 119 - DNA, 21 bp, Artificial Sequence
  Synthetic
gacattgccatgccagctaag

SEQ ID NO: 120 - DNA, 20 bp, Artificial Sequence
  Synthetic
tgtggtgccttttcggctta

SEQ ID NO: 121 - DNA, 20 bp, Artificial Sequence
  Synthetic
atgaccctgcgccttatttg

SEQ ID NO: 122 - DNA, 19 bp, Artificial Sequence
  Synthetic
ttggtcaccttcagcttgg

SEQ ID NO: 123 - DNA, 20 bp, Artificial Sequence
  Synthetic
ggggttcctccatctaggtc

SEQ ID NO: 124 - DNA, 20 bp, Artificial Sequence
  Synthetic
atgctgagggaaccaaaagc

SEQ ID NO: 125 - DNA, 22 bp, Artificial Sequence
  Synthetic
catggtcctgctggagttcgtg

SEQ ID NO: 126 - DNA, 19 bp, Artificial Sequence
  Synthetic
ttggtcaccttcagcttgg

SEQ ID NO: 127 - DNA, 20 bp, Artificial Sequence
  Synthetic
aagtggccttctagagctgg

SEQ ID NO: 128 - DNA, 20 bp, Artificial Sequence
  Synthetic
ctagctagagtgcttggggc

SEQ ID NO: 129 - DNA, 22 bp, Artificial Sequence
  Synthetic
ctgagcacgtagggataacagg

SEQ ID NO: 130 - DNA, 20 bp, Artificial Sequence
  Synthetic
tcaaatcctcgcatttgggc

SEQ ID NO: 131 - DNA, 44 bp, Artificial Sequence; identical to SEQ ID NO: 91
  Synthetic

SEQ ID NO: 132 - DNA, 41 bp, Artificial Sequence; identical to SEQ ID NO: 92
  Synthetic

SEQ ID NO: 133 - DNA, 47 bp, Artificial Sequence
  Synthetic
ctacacgacgctcttccgatctactgttgtgttgagtaacatatacc

SEQ ID NO: 134 - DNA, 43 bp, Artificial Sequence
  Synthetic
agacgtgtgctcttccgatctaacaagacaccaggaaaagaca

SEQ ID NO: 135 - DNA, 45 bp, Artificial Sequence
  Synthetic
ctacacgacgctcttccgatctcaattaagtgtgttgttgtgggc

SEQ ID NO: 136 - DNA, 46 bp, Artificial Sequence
  Synthetic
agacgtgtgctcttccgatctagtatctgatcagcttaccttttct

SEQ ID NO: 137 - DNA, 43 bp, Artificial Sequence
  Synthetic
ctacacgacgctcttccgatctttaagtgtgttgttgtgggcg

SEQ ID NO: 138 - DNA, 43 bp, Artificial Sequence
  Synthetic
agacgtgtgctcttccgatctctacagtagtgggacaggaagc

SEQ ID NO: 139 - DNA, 42 bp, Artificial Sequence; identical to SEQ ID NO: 97
  Synthetic

SEQ ID NO: 140 - DNA, 40 bp, Artificial Sequence; identical to SEQ ID NO: 98
  Synthetic

SEQ ID NO: 141 - DNA, 43 bp, Artificial Sequence; identical to SEQ ID NO: 103
  Synthetic

SEQ ID NO: 142 - DNA, 42 bp, Artificial Sequence; identical to SEQ ID NO: 104
  Synthetic

SEQ ID NO: 143 - DNA, 43 bp, Artificial Sequence; identical to SEQ ID NO: 105
  Synthetic

SEQ ID NO: 144 - DNA, 42 bp, Artificial Sequence; identical to SEQ ID NO: 106
  Synthetic

SEQ ID NO: 145 - DNA, 41 bp, Artificial Sequence; identical to SEQ ID NO: 107
  Synthetic

SEQ ID NO: 146 - DNA, 44 bp, Artificial Sequence; identical to SEQ ID NO: 108
  Synthetic

SEQ ID NO: 147 - DNA, 32 bp, Artificial Sequence
  Synthetic
actgagcacgtgatggcctcacatttgggcag

SEQ ID NO: 148 - DNA, 38 bp, Artificial Sequence
  Synthetic
actgagcacgtagggataacagggtaatatttgggcag

SEQ ID NO: 149 - DNA, 20 bp, Artificial Sequence
  Synthetic
actgagcacgatttgggcag

SEQ ID NO: 150 - DNA, 13 bp, Artificial Sequence
  Synthetic
cacgtttgggcag

SEQ ID NO: 151 - DNA, 62 bp, Artificial Sequence
  Synthetic
actgagcacgtagggataacagggtaatgcaccgactcggtcccactttttcatttgggcag

SEQ ID NO: 152 - DNA, 35 bp, Artificial Sequence
  Synthetic
actgagctagggataacagggtaatatttgggcag

SEQ ID NO: 153 - DNA, 34 bp, Artificial Sequence
  Synthetic
tcgagcgccaccatggccctgtgagcaagggcga

SEQ ID NO: 154 - DNA, 28 bp, Artificial Sequence
  Synthetic
tcgagcgccaccatggtgagcaagggcg

SEQ ID NO: 155 - DNA, 25 bp, Artificial Sequence
  Synthetic
tcgagcgccatggtgagcaagggcg

SEQ ID NO: 156 - DNA, 17 bp, Artificial Sequence
  Synthetic
tcttctcggccaatgtt

SEQ ID NO: 157 - DNA, 27 bp, Artificial Sequence
  Synthetic
tcttctcggcagcatgccaccaatgtt

SEQ ID NO: 158 - DNA, 27 bp, Artificial Sequence
  Synthetic
tcctctcggcagcatgccaccaatgtt

SEQ ID NO: 159 - DNA, 27 bp, Artificial Sequence
  Synthetic
tcttctcggcagcatgccaccaacgtt

SEQ ID NO: 160 - DNA, 26 bp, Artificial Sequence
  Synthetic
tctactcggcagcatgccaccatgtt

SEQ ID NO: 161 - DNA, 57 bp, Artificial Sequence
  Synthetic
ttggggcccagactgagcacgggataacagggtaatatttgggcaggtgatcaatgc

SEQ ID NO: 162 - DNA, 62 bp, Artificial Sequence
  Synthetic
ttggggcccagactgagcacggctagggataacagggtaatatttgggcaggtgatcaatgc